(19) United States
(12) Patent Application Publication
(10) Pub. No.: US 2022/0354623 A1
(43) Pub. Date: Nov. 10, 2022

(54) METHOD AND APPARATUS FOR MAPPING TOOTH SURFACES

(71) Applicant: Cyberdontics (USA), Inc., San Francisco, CA (US)

(72) Inventors: Christopher John CIRIELLO, San Francisco, CA (US); Scott Howard PHILLIPS, Victoria (CA); James JACKSON, Victoria (CA); Kenneth MACCALLUM, Victoria (CA); Nathan John MULLER, Victoria (CA); Stephen Bruce LOCKE, Ajax (CA); Ryan Lucas FIELD, Toronto (CA); Jacob ROSEN, Los Angeles, CA (US)

(21) Appl. No.: 17/813,789

(22) Filed: Jul. 20, 2022

Related U.S. Application Data
(63) Continuation of application No. PCT/US2021/015555, filed on Jan. 28, 2021.
(60) Provisional application No. 62/967,419, filed on Jan. 29, 2020.
(60) Provisional application No. 63/120,487, filed on Dec. 2, 2020.
(60) Provisional application No. 63/122,809, filed on Dec. 8, 2020.

(51) Int. Cl. A61C 9/00; A61C 13/34; A61B 1/24; A61B 5/00

(52) U.S. Cl. CPC A61C 9/0053; A61C 9/008; A61C 13/34; A61B 1/24; A61B 5/0002

(57) ABSTRACT

Provided herein are platforms and methods for mapping a three-dimensional (3D) dental anatomy of a subject.

### CROSS-REFERENCE

This application is a continuation of International Application No. PCT/US2021/015555, which claims the benefit of U.S. Provisional Application No. 62/967,419, filed Jan. 29, 2020, U.S. Provisional Application No. 63/120,487, filed Dec. 2, 2020, and U.S. Provisional Application No. 63/122,809, filed Dec. 8, 2020, which are hereby incorporated by reference in their entirety herein.

### BACKGROUND

Surface and subsurface dental imaging has been advantageous for diagnosis, and for performing and/or planning treatments. Improved accuracy increases the reliability and efficacy of such diagnoses and treatments.

### SUMMARY

One aspect provided herein is platform for mapping a three-dimensional (3D) dental anatomy of a subject, the platform comprising: a non-contact mapping system comprising: a non-contact sensor capturing a first morphology data of at least a portion of the mouth of the subject; and a sensor transmission module transmitting the first morphology data; a contact mapping system comprising: a probe comprising: a probe orientation sensor measuring an orientation of the probe; a probe contact surface; and a probe transmission module transmitting the orientation of the probe; and a datum comprising: a datum fastener for mounting to the subject; a datum orientation sensor measuring an orientation of the datum; and a probe transmission module transmitting the orientation of the datum; and a modeling system receiving: the first morphology data; the orientation of the probe; and the orientation of the datum; wherein the modeling system determines a second morphology data based on the orientation of the probe and the orientation of the datum; and wherein the modeling system determines the 3D dental anatomy based on the first morphology data and the second morphology data.

Another aspect provided herein is a platform for mapping a three-dimensional (3D) dental anatomy of a subject, the platform comprising: a non-contact mapping system comprising: a non-contact sensor capturing a first morphology data of at least a portion of the mouth of the subject; and a sensor transmission module transmitting the first morphology data; a contact mapping system comprising: a probe comprising: a probe orientation sensor measuring an orientation of the probe; a probe contact surface; and a probe transmission module transmitting the orientation of the probe; and a modeling system receiving: the first morphology data; and the orientation of the probe; wherein the modeling system determines the 3D dental anatomy based on the first morphology data and the orientation of the probe.

Another aspect provided herein is a platform for mapping a three-dimensional (3D) dental anatomy of a subject, the platform comprising: a non-contact mapping system comprising: a non-contact mapping system comprising: a first non-contact sensor capturing a first portion of a first morphology data of at least a portion of the mouth of the subject; a second non-contact sensor capturing a second portion of the first morphology data of at least a portion of the mouth of the subject; and a sensor transmission module transmitting the primary first morphology data; a modeling system receiving: the first morphology data; and wherein the modeling system determines the 3D dental anatomy based on the primary first morphology data.

In some embodiments, the contact mapping system further comprises a datum comprising a datum fastener for mounting to the subject. In some embodiments, the datum further comprises a datum orientation sensor measuring an orientation of the datum and a probe transmission module transmitting the orientation of the datum. In some embodiments, the modeling system further receives the orientation of the datum. In some embodiments, the modeling system determines a second morphology data based on the orientation of the probe and the orientation of the datum. In some embodiments, the modeling system determines the 3D dental anatomy based on the first morphology data and the second morphology data. In some embodiments, the datum orientation sensor comprises an accelerometer, a tilt sensor, a gyroscope, a GPS sensor, a distance sensor, a RADAR, a magnet, a radio frequency generator, a radio frequency receiver, or any combination thereof. In some embodiments, the datum orientation sensor is located within at most about 2 inches from the center of mass of the datum. In some embodiments, the orientation of the datum comprises a rotation of the datum about one or more axes, a translation of the datum in one or more directions, a rotational velocity of the datum about one or more axes, a translational velocity of the datum in one or more directions, an angular acceleration of the datum about one or more axes, a translational acceleration of the datum in one or more directions, or any combination thereof. In some embodiments, the datum fastener comprises a clip, an adhesive, a clamp, a band, a tie, or any combination thereof. In some embodiments, the datum fastener rigidly and removably mounts to the subject. In some embodiments, the datum fastener mounts to a tooth of the subject, the jaw of the subject, or both. In some embodiments, the datum fastener mounts outside the subject's mouth. In some embodiments, the datum further comprises a datum fiducial visible to the non-contact sensor. In some embodiments, first morphology data comprises the datum fiducial. In some embodiments, the non-contact mapping system comprises two or more non-contact sensors. In some embodiments, the first morphology comprises a visible surface morphology, a subsurface occluded morphology, or both. In some embodiments, the subsurface occluded morphology comprises a tooth pulp, a muscle, a nerve, a blood vessel, or any combination thereof. In some embodiments, the non-contact sensor comprises a 3D scanner, a LIDAR, a RADAR, a laser, a camera, a microscope, an optical coherence tomogram (OCT), a confocal laser scanning microscope (CLSM), or any combination thereof. In some embodiments, the OCT comprises a time-domain OCT, a Fourier-domain OCT, a swept-source OCT, or any combination thereof. In some embodiments, the camera comprises an endoscopic camera. In some embodiments, the endoscopic camera comprises an illumination source. In some embodiments, the microscope comprises a confocal laser scanning microscope, a multi-photon microscope, or both. In some embodiments, the confocal laser scanning microscope, the multi-photon microscope, or both captures an image of the oral cavity of the subject and a fluorescence within the oral cavity of the subject. In some embodiments, the non-contact sensor captures the first morphology by point-by-point scanning, line scanning, raster scanning, full-field scanning, or any combination thereof. In some embodiments, the non-contact mapping system further comprises a switch to initiate the capture of the first morphological data, terminate the capture of the first morphological data, or both. In some embodiments, the non-contact mapping system further comprises a reference fiducial rigidly and removably mountable to the subject. In some embodiments, the non-contact sensor further captures the reference fiducial, and wherein the first morphology data further comprises a location of the reference fiducial, orientation of the reference fiducial, or both. In some embodiments, the contact mapping system comprises two or more contact sensors. In some embodiments, the probe orientation sensor comprises an accelerometer, a tilt sensor, a gyroscope, a GPS sensor, a distance sensor, a RADAR, a magnet, a radio frequency generator, a radio frequency receiver, or any combination thereof. In some embodiments, the probe orientation sensor is located within at most about 2 inches from the center of mass of the probe. In some embodiments, the orientation of the probe comprises a rotation of the probe about one or more axes, a translation of the probe in one or more directions, a rotational velocity of the probe about one or more axes, a translational velocity of the probe in one or more directions, an angular acceleration of the probe about one or more axes, a translational acceleration of the probe in one or more directions, or any combination thereof. In some embodiments, wherein the probe comprises a periodontal endoscope. In some embodiments, the probe contact surface comprises a sub-gingival probe contact surface. In some embodiments, the probe contact surface is generally acute. In some embodiments, the probe contact surface is rounded. In some embodiments, at least a portion of the probe contact surface is rigid. In some embodiments, at least a portion of the probe contact surface is flexible. In some embodiments, at least a portion of the probe contact surface is removable from the probe. In some embodiments, the probe further comprises one or more of: a force sensor measuring a probe force between the probe contact surface and a dental surface of the subject; and a touch sensor determining if the distal probe contacts the dental surface of the subject. In some embodiments, the probe transmission module further transmits the probe force, the contact determination, or both. In some embodiments, the modeling system further determines the 3D dental anatomy based on the probe force, the contact determination, or both. In some embodiments, a center axis of at least a portion of the probe contact surface is parallel to a center axis of at least a portion of the handle. In some embodiments, a center axis of at least a portion of the probe contact surface is askew from a center axis of at least a portion of the handle. In some embodiments, the probe further comprises a probe fiducial visible to the non-contact sensor. In some embodiments, first morphology data comprises the probe fiducial.

In some embodiments, the probe further comprises a probe light sensor, and wherein the platform further comprises a pulsed light emitter. In some embodiments, a beam of light emitted by the pulsed light emitter translates, rotates, or both, with respect to the probe light sensor. In some embodiments, the probe transmission module further transmits the sensed probe light. In some embodiments, the modeling system further determines the 3D dental anatomy based on the sensed probe light. In some embodiments, the datum further comprises a datum light sensor, and wherein the platform further comprises a pulsed light emitter. In some embodiments, a beam of light emitted by the pulsed light emitter translates, rotates, or both, with respect to the datum light sensor. In some embodiments, the datum transmission module further transmits the sensed datum light. In some embodiments, the modeling system further determines the 3D dental anatomy based on the sensed datum light. In some embodiments, the platform further comprises an actuator coupled to the non-contact sensor. In some embodiments, the platform further comprises an actuator coupled to the probe. In some embodiments, the probe comprises a first coupling that removably connects to the actuator. In some embodiments, the non-contact sensor comprises a second coupling that removably connects to the actuator. In some embodiments, the non-contact sensor captures a first morphology at a first location, wherein the actuator orients the non-contact sensor to a second location different from the first location, and wherein the non-contact sensor captures a third morphology at the second location. In some embodiments, the platform further comprises a encoder measuring a position of the non-contact mapping system. In some embodiments, the platform further comprises a encoder measuring a position of the probe. In some embodiments, the first morphology is based on a measurement by the passive encoder. In some embodiments, the second morphology is based on a measurement by the passive encoder. In some embodiments, the encoder is a rotational encoder. In some embodiments, the encoder is a translational encoder. In some embodiments, the platform further comprises a mouth coupling device removably coupling the non-contact mapping to the mouth of the patient. In some embodiments, the modeling system further determines the 3D dental anatomy based on a historical morphology data of the subject. In some embodiments, the modeling system normalizes the first morphology data. In some embodiments, the modeling system normalizes the second morphology data. In some embodiments, the first morphology data is normalized by a first machine learning algorithm. In some embodiments, the second morphology data is normalized by the first machine learning. In some embodiments, the modeling system further determines the 3D dental anatomy based on a predetermined anatomy landmark. In some embodiments, the modeling system determines the 3D dental anatomy by triangulation, confocal imaging, stereophotogrammetry, or any combination thereof. In some embodiments, the anatomy landmark comprises a gum margin, a marked tooth, or both. In some embodiments, the modeling system further determines the 3D dental anatomy by applying a second machine learning algorithm. In some embodiments, the modeling system further determines an anatomy classification based on the first morphology data and the second morphology data. In some embodiments, the anatomy classification comprises a tooth classification, a gum classification, a lip classification, a cheek classification, a tongue classification, or any combination thereof. In some embodiments, the modeling system determines the anatomy classification by applying a third machine learning algorithm. In some embodiments, the modeling system further extrapolates the 3D dental anatomy. In some embodiments, the modeling system extrapolates the 3D dental anatomy using a fourth machine learning algorithm. In some embodiments, the modeling system further interpolates the 3D dental anatomy. In some embodiments, the modeling system interpolates the 3D dental anatomy using a fifth machine learning algorithm. In some embodiments, the platform further comprises a dental effector performing a dental surgery based at least in part on the 3D dental anatomy of the subject. In some embodiments, the dental anatomy is of a tooth, a jaw, a gum, a lingual tooth surface, a subgingival surface, an interproximal gap, or any combination thereof. In some embodiments, the first morphology data, the second morphology data, the 3D dental anatomy, or any combination thereof comprises a point cloud, a mesh, a surface model, or any combination thereof. In some embodiments, the sensor transmission module, the probe transmission module, or both comprise a Bluetooth transmitter, a Wi-Fi transmitter, a cellular transmitter, a wired transmitter, an optical transmitter, or any combination thereof.

Another aspect provided herein is a method for mapping a three-dimensional (3D) dental anatomy of a subject, the method comprising: capturing, by non-contact sensing, a first morphology data of at least a portion of the mouth of the subject; transmitting, the first morphology data; measuring an orientation of a probe while a probe contact surface of the probe contacts the mouth of the subject; transmitting, the orientation of the probe; measuring, by a datum orientation sensor mounted to the subject, an orientation of the datum; transmitting, the orientation of the datum; determining a second morphology data based on the orientation of the probe and the orientation of the datum; and determining the 3D dental anatomy based on the first morphology data and the second morphology data.

Another aspect provided herein is a method for mapping a three-dimensional (3D) dental anatomy of a subject, the method comprising: capturing, by a non-contact sensor, a first morphology data of at least a portion of the mouth of the subject; transmitting, by a sensor transmission module, the first morphology data; measuring, by a probe orientation sensor having a probe contact surface, an orientation of a probe while the probe contact surface contacts the mouth of the subject; transmitting, by a probe transmission module, the orientation of the probe; determining the 3D dental anatomy based on the first morphology data and the orientation of the probe.

Another aspect provided herein is a method for mapping a three-dimensional (3D) dental anatomy of a subject, the method comprising: capturing, by a first non-contact sensor, a first portion of a first morphology data of at least a portion of the mouth of the subject; capturing, by a second non-contact sensor, a second portion of the first morphology data of at least a portion of the mouth of the subject; transmitting, by a sensor transmission module, the first morphology data; determining the 3D dental anatomy based on the first morphology data.

In some embodiments, the method further comprises measuring, by a datum orientation sensor, an orientation of the datum; and transmitting, by a probe transmission module, the orientation of the datum. In some embodiments, the second morphology data is determined based on the orientation of the probe and the orientation of the datum. In some embodiments, the 3D dental anatomy is determined based on the first morphology data and the second morphology data. In some embodiments, the datum orientation sensor comprises an accelerometer, a tilt sensor, a gyroscope, a GPS sensor, a distance sensor, a RADAR, a magnet, a radio frequency generator, a radio frequency receiver, or any combination thereof. In some embodiments, the datum orientation sensor is located within at most about 2 inches from the center of mass of the datum. In some embodiments, the orientation of the datum comprises a rotation of the datum about one or more axes, a translation of the datum in one or more directions, a rotational velocity of the datum about one or more axes, a translational velocity of the datum in one or more directions, an angular acceleration of the datum about one or more axes, a translational acceleration of the datum in one or more directions, or any combination thereof. In some embodiments, the method further comprises mounting the datum to the subject with a datum fastener. In some embodiments, mounting the datum to the subject comprises rigidly and removably mounting the datum to the subject. In some embodiments, mounting the datum to the subject comprises mounting the datum to a tooth of the subject, the jaw of the subject, or both. In some embodiments, mounting the datum to the subject comprises mounting the datum outside the subject's mouth. In some embodiments, the method further comprises capturing, by the non-contact sensor, a datum fiducial on the datum. In some embodiments, first morphology data comprises the datum fiducial. In some embodiments, the non-contact sensor comprises a 3D scanner, a LIDAR, a RADAR, a laser, a camera, a microscope, an optical coherence tomogram (OCT), a confocal laser scanning microscope (CLSM), or any combination thereof. In some embodiments, the OCT comprises a time-domain OCT, a Fourier-domain OCT, a swept-source OCT, or any combination thereof. In some embodiments, the camera comprises an endoscopic camera. In some embodiments, the endoscopic camera comprises an illumination source. In some embodiments, the microscope comprises a confocal laser scanning microscope, a multi-photon microscope, or both. In some embodiments, the confocal laser scanning microscope, the multi-photon microscope, or both, capture an image of a fluorescence within the oral cavity of the subject. In some embodiments, the method further comprises applying the fluorescence to the oral cavity of the subject. In some embodiments, the first morphology comprises a visible surface morphology, a subsurface occluded morphology, or both. In some embodiments, the subsurface occluded morphology comprises a tooth pulp, a muscle, a nerve, a blood vessel, or any combination thereof. In some embodiments, capturing the first morphology comprises point-by-point scanning, line scanning, raster scanning, full-field scanning, or any combination thereof. In some embodiments, capturing the first morphology comprises activating a switch to initiate the capture of the first morphological data, terminate the capture of the first morphological data, or both. In some embodiments, the method further comprises mounting a reference fiducial to the subject. In some embodiments, the first morphology data further comprises a location of the reference fiducial, orientation of the reference fiducial, or both. In some embodiments, the probe orientation sensor comprises an accelerometer, a tilt sensor, a gyroscope, a GPS sensor, a distance sensor, a RADAR, a magnet, a radio frequency generator, a radio frequency receiver, or any combination thereof. In some embodiments, the probe orientation sensor is located within at most about 2 inches from the center of mass of the probe. In some embodiments, the orientation of the probe comprises a rotation of the probe about one or more axes, a translation of the probe in one or more directions, a rotational velocity of the probe about one or more axes, a translational velocity of the probe in one or more directions, an angular acceleration of the probe about one or more axes, a translational acceleration of the probe in one or more directions, or any combination thereof. In some embodiments, the probe contact surface is generally acute. In some embodiments, wherein the probe comprises a periodontal endoscope. In some embodiments, the probe contact surface comprises a sub-gingival probe contact surface. In some embodiments, the probe contact surface is rounded. In some embodiments, at least a portion of the probe contact surface is rigid. In some embodiments, at least a portion of the probe contact surface is flexible. In some embodiments, the method further comprises one or more of: measuring, by a force sensor, a probe force between the probe contact surface and a dental surface of the subject; and determining, by a touch sensor, if the distal probe contacts the dental surface of the subject. In some embodiments, the method further comprises transmitting, by the transmission module, the probe force, the contact determination, or both. In some embodiments, the 3D dental anatomy is further based on the probe force, the contact determination, or both. In some embodiments, a center axis of at least a portion of the probe contact surface is parallel to a center axis of at least a portion of the handle. In some embodiments, a center axis of at least a portion of the probe contact surface is askew from a center axis of at least a portion of the handle. In some embodiments, the probe further comprises a probe fiducial visible to the non-contact sensor. In some embodiments, first morphology data comprises the probe fiducial. In some embodiments, the probe further comprises a probe light sensor, and wherein the method further comprises emitting a light beam by a pulsed light emitter. In some embodiments, the method further comprises translating the light beam, rotating the light beam, or both, with respect to the probe light sensor. In some embodiments, the method further comprises transmitting, by the probe transmission module, the sensed probe light. In some embodiments, 3D dental anatomy is further based on the sensed probe light. In some embodiments, the datum further comprises a datum light sensor, and wherein the method further comprises emitting a light beam by a pulsed light emitter. In some embodiments, the method further comprises translating the light beam, rotating the light beam, or both, with respect to the datum light sensor. In some embodiments, the method further comprises, by the datum transmission module, the sensed datum light. In some embodiments, the 3D dental anatomy is further based on the sensed datum light. In some embodiments, the method further comprises orienting, by an actuator, the non-contact mapping system. In some embodiments, the method further comprises orienting, by an actuator, the probe. In some embodiments, the method further comprises capturing the first morphology at a first location, orienting, by the actuator, the non-contact sensor to a second location different from the first location, and capturing, by the non-contact sensor, a third morphology at the second location. In some embodiments, the method further comprises measuring, by an encoder, a position of the non-contact mapping system. In some embodiments, the method further comprises measuring, by an encoder, a position of the probe. In some embodiments, the first morphology is based on a measurement by the passive encoder. In some embodiments, the second morphology is based on a measurement by the passive encoder. In some embodiments, the encoder is a rotational encoder. In some embodiments, the encoder is a translational encoder. In some embodiments, the method further comprises coupling the non-contact mapping sensor to the mouth of the subject. In some embodiments, the 3D dental anatomy is further based on a historical morphology data of the subject. In some embodiments, the method further comprises normalizing the first morphology data. In some embodiments, the method further comprises normalizing the second morphology data. In some embodiments, the first morphology data is normalized by a first machine learning algorithm. In some embodiments, the first morphology data is normalized by a first machine learning algorithm. In some embodiments, the 3D dental anatomy is further based on a predetermined anatomy landmark. In some embodiments, the 3D dental anatomy is determined by triangulation, confocal imaging, stereophotogrammetry, or any combination thereof. In some embodiments, the anatomy landmark comprises a gum margin, a marked tooth, or both. In some embodiments, the 3D dental anatomy is determined by applying a second machine learning algorithm. In some embodiments, the method further comprises determining an anatomy classification based on the first morphology data and the second morphology data. In some embodiments, the anatomy classification comprises a tooth classification, a gum classification, a lip classification, a cheek classification, a tongue classification, or any combination thereof. In some embodiments, the anatomy classification is determined by applying a third machine learning algorithm. In some embodiments, the method further comprises extrapolating the 3D dental anatomy. In some embodiments, extrapolating the 3D dental anatomy is performed by a fourth machine learning algorithm. In some embodiments, the modeling system further interpolates the 3D dental anatomy. In some embodiments, the modeling system interpolates the 3D dental anatomy using a fifth machine learning algorithm. In some embodiments, the method further comprises performing a dental surgery based at least in part on the 3D dental anatomy of the subject. In some embodiments, the dental anatomy is of a tooth, a jaw, a gum, a lingual tooth surface, a subgingival surface, an interproximal gap, or any combination thereof. In some embodiments, the first morphology data, the second morphology data, the 3D dental anatomy, or any combination thereof comprises a point cloud, a mesh, a surface model, or any combination thereof. In some embodiments, the sensor transmission module, the probe transmission module, or both comprise a Bluetooth transmitter, a Wi-Fi transmitter, a cellular transmitter, a wired transmitter, an optical transmitter, or any combination thereof.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a diagram of a first exemplary platform for mapping a three-dimensional (3D) dental anatomy, per one or more embodiments herein;

FIG. 2A shows a diagram of an exemplary probe, per one or more embodiments herein;

FIG. 2B shows a diagram of an exemplary datum, per one or more embodiments herein;

FIG. 3 shows an illustration of an exemplary non-contact sensor, per one or more embodiments herein;

FIG. 4 shows an illustration of an exemplary probe contacting a sub-gingival dental surface of a subject, per one or more embodiments herein;

FIG. 5 shows an illustration of an exemplary method of extrapolating a 3D dental anatomy, per one or more embodiments herein;

FIG. 6A shows a front view of an exemplary segmented 3D dental anatomy and a vertical projection extending therefrom, per one or more embodiments herein;

FIG. 6B shows front-bottom perspective view of an exemplary segmented 3D dental anatomy and a vertical projection extending therefrom, per one or more embodiments herein;

FIG. 6C shows a front view image of the exemplary segmented 3D dental anatomy, a vertical projection, and a model of an existing tooth, per one or more embodiments herein;

FIG. 6D shows a front-bottom perspective view image of the exemplary segmented 3D dental anatomy, the vertical projection and an exemplary gum surface, per one or more embodiments herein;

FIG. 7A shows a front view of an exemplary segmented 3D dental anatomy and a gradient projection extending therefrom, per one or more embodiments herein;

FIG. 7B shows front-bottom perspective view of an exemplary segmented 3D dental anatomy and a gradient projection extending therefrom, per one or more embodiments herein;

FIG. 7C shows a front view image of the exemplary segmented 3D dental anatomy, a gradient projection, and a model of an existing tooth, per one or more embodiments herein;

FIG. 7D shows a front-bottom perspective view image of an exemplary gum surface, per one or more embodiments herein;

FIG. 8 shows a diagram of a second exemplary platform for mapping a three-dimensional (3D) dental anatomy comprising an optical coherence tomography (OCT) non-contact sensor, per one or more embodiments herein;

FIG. 9 shows a diagram of a third exemplary platform for mapping a 3D dental anatomy comprising an OCT non-contact sensor, per one or more embodiments herein;

FIG. 10 shows a diagram of a fourth exemplary platform for mapping a 3D dental anatomy wherein the probe comprises a photodetector sensor, per one or more embodiments herein; and

FIG. 11 shows a non-limiting example of a computing device; in this case, a device with one or more processors, memory, storage, and a network interface.

### DETAILED DESCRIPTION

While conventional three-dimensional (3D) intraoral scanners (IOS) have been developed to image visible dental anatomy, such technologies are usable to capture morphologies of subgingival and interproximal regions of the teeth that are hidden from view.

Further, while capturing optical coherence tomography (OCT) from a fixed location can image such occluded surfaces below the gumline, such methods may only capture a portion of such geometry due to a limited transverse field-of-view, and fixed depth measurement. As such, provided herein are methods, systems, and platforms for translating an OCT probe. Further, provided herein are methods, systems, and platforms capable of imaging a greater volume of occluded dental surfaces by combining OCT measurements as captured from multiple views and depths to form a more complete and accurate three-dimensional (3D) image.

### Platforms for Mapping a Three-Dimensional (3D) Dental Anatomy

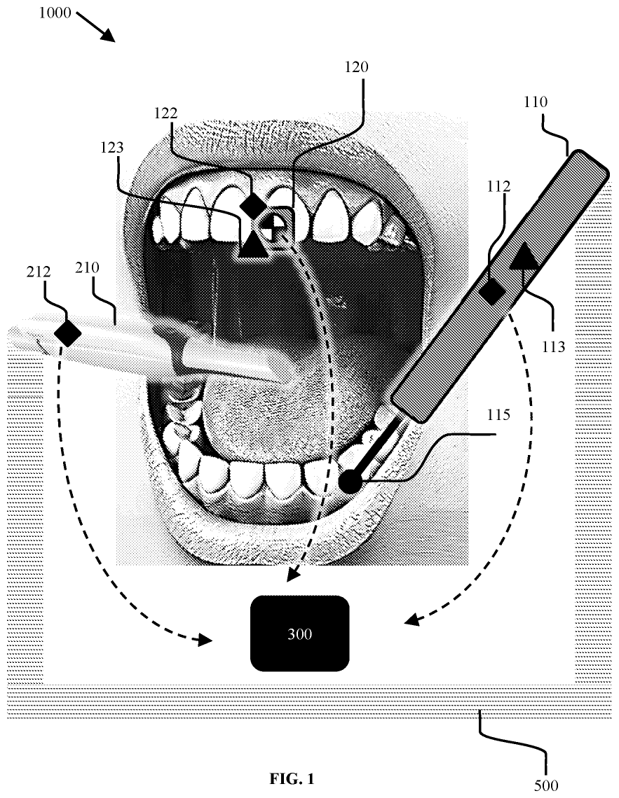

One aspect provided herein, per FIG. 1, is a first platform 1000 for mapping a three-dimensional (3D) dental anatomy of a subject. In some embodiments, the platform 1000 comprises a non-contact mapping system 300, a contact mapping system 300, and a modeling system. In some embodiments, the non-contact mapping system 300 comprises a non-contact sensor 210 and a sensor transmission module 212. In some embodiments, the non-contact mapping system 300 comprises two or more non-contact sensors 210. In some embodiments, the non-contact mapping system 300 comprises 2, 3, 4, 5, 6, 7, 8, 9, 10 or more non-contact sensors 210. In some embodiments, the two or more non-contact sensors 210 are rigidly coupled. In some embodiments, at least one non-contact sensor 210 moves with respect to another non-contact sensor 210. In some embodiments, the non-contact sensor captures a first morphology data of at least a portion of the mouth of the subject In some embodiments, the first morphology data from two or more non-contact sensors 210 are combined using triangulation, stereophotogrammetry, or both. In some embodiments, the non-contact sensor 210 and the sensor transmission module 212 are integrated into a single component. In some embodiments, the non-contact sensor 210 and the sensor transmission module 212 are separate and/or distinct.

In some embodiments, the contact mapping system 300 comprises a probe and a datum 120. In some embodiments, the contact mapping system 300 does not comprise the datum 120 In some embodiments, the contact mapping system 300 comprises two or more contact sensors 110. In some embodiments, the non-contact mapping system 300 comprises 2, 3, 4, 5, 6, 7, 8, 9, 10 or more non-contact sensors 110. In some embodiments, the probe 110 comprises a probe orientation sensor 213 measuring an orientation of the probe 110, a probe contact surface 115, and a probe transmission module 112 transmitting the orientation of the probe 110. In some embodiments, the datum 120 comprises a datum 120 fastener, a datum orientation sensor 123, and a datum transmission module 122.

In some embodiments, the dental anatomy is a tooth, a jaw, a gum, a lingual tooth surface, a subgingival surface, an interproximal gap, or any combination thereof. In some embodiments, the first morphology data, the second morphology data, the 3D dental anatomy, or any combination thereof comprise a point cloud, a mesh, a surface model, or any combination thereof. In some embodiments, the first morphology data, the second morphology data, the 3D dental anatomy, or any combination thereof comprise a 3D surface data, a 3D volumetric data, or both. In some embodiments, the first morphology comprises a visible surface morphology, a subsurface occluded morphology, or both. In some embodiments, the visible surface morphology comprises a tooth surface, a gum surface, a cheek surface, a tongue surface, or any combination thereof. In some embodiments, the subsurface occluded morphology represents a morphology within a dental tissue. In some embodiments, the subsurface occluded morphology comprises a tooth pulp, a muscle, a nerve, a blood vessel, or any combination thereof.

In some embodiments, the platform 1000 further comprises an actuator 500 coupled to the non-contact sensor 210, the probe 110, or both. In some embodiments, the platform 1000 further comprises a dental effector performing a dental surgery and/or a dental procedure based on the 3D dental anatomy of the subject. In some embodiments, the 3D dental anatomy is used at least in part to plan a treatment method. In some embodiments, the dental surgery comprises an apicoectomy, extraction, fiberotomy, implantation, maxillofacial surgery, periodontal surgery, prosthodontal surgery, pulpectomy, pulpotomy, or a root canal treatment. In some embodiments, the dental procedure comprises an orthodontic procedure, a veneer procedure, or a cleaning procedure. In some embodiments, the dental effector comprises a drill, a laser, a scalpel, or any combination thereof.

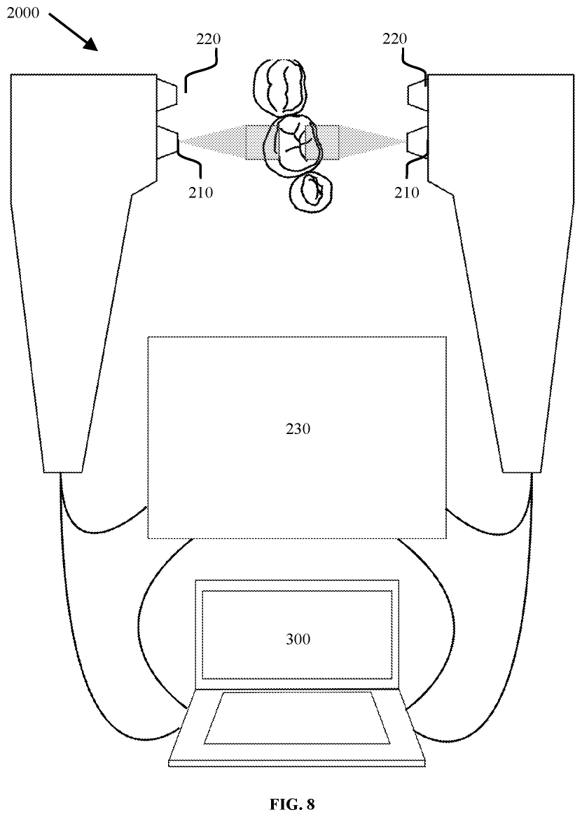

FIG. 8 shows a diagram of a second exemplary platform 2000 for mapping a three-dimensional (3D) dental anatomy comprising two first non-contact sensors 210 and two second non-contact sensors 220. In some embodiments, as shown, the tooth of the subject is positioned between each pair of first non-contact sensors 210 and each pair of second non-contact sensors 220. In such an embodiment, the non-contact sensor system captures a first morphology data encompassing both inner and outer surfaces of the tooth of the subject. In some embodiments, the first non-contact sensor 210 comprises an optical coherence tomography (OCT) non-contact sensor, wherein an OCT system 230 provides the first non-contact sensors 210 with light, power, or both. In some embodiments, the OCT system 230 connects to the first non-contact sensors 210 wirelessly. In some embodiments, the OCT system 230 connects to the first non-contact sensors 210 by an optic cable. In some embodiments, the optic cable is sufficiently flexible such that the platform 2000 can be moved without imparting force on the first non-contact sensors 210 and the second non-contact sensors 220. In some embodiments, the first non-contact sensors 210 and the second non-contact sensors 220 transmit the first morphology to the modeling system 300. In some embodiments, the first non-contact sensors 210 and the second non-contact sensors 220 transmit the first morphology to the OCT system 230. In some embodiments, the OCT system 230 transmits the first morphology to the modeling system 300. In some embodiments, the OCT system 230 is integrated into the modeling system 300. In some embodiments, the OCT system 230 is integrated into the first non-contact sensors 210, the second non-contact sensors 220, or both.

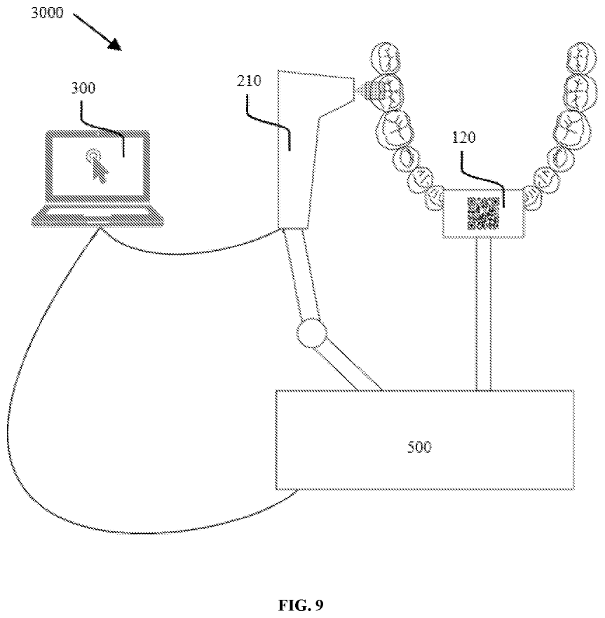

FIG. 9 shows a diagram of a third exemplary platform for mapping a 3D dental anatomy 3000. In some embodiments, the third exemplary platform for mapping a 3D dental anatomy 3000 comprises a single non-contact sensor 210, a datum 120 having a fiducial, and an actuator 500. As shown, in some embodiments, the datum 120 is coupled to a tooth of the subject. As shown, in some embodiments, the actuator 500 is coupled to the non-contact sensor 210. In some embodiments, the actuator 500 is further coupled to the tooth of the subject, the datum 120, or both. In some embodiments, the modeling system 300 directs the motion of the actuator.

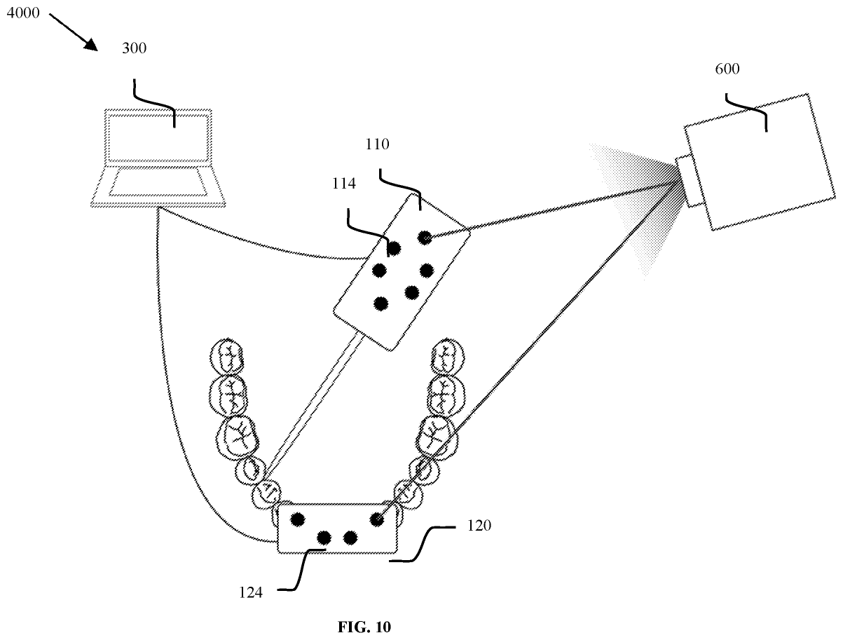

FIG. 10 shows a diagram of a fourth exemplary platform 4000 for mapping a 3D dental anatomy comprising a pulsed light emitter 600, wherein the probe 110 comprises a probe light sensor 114, and wherein the datum 120 comprises a datum light sensor 124. In some embodiments, a beam of light emitted by the pulsed light emitter 600 translates, rotates, or both, with respect to the datum light sensor 124. In some embodiments, the datum transmission module 122 further transmits the sensed datum 120 light. In some embodiments, the datum light sensor 124 comprises a photodiode, a photodetector, or both. In some embodiments, the pulsed light emitter 600 is a laser, a light emitting diode (LED), or both. In some embodiments, the orientation of the datum 120 is based on the light sensed by the light sensor. In some embodiments, the pulsed light emitter 600 emits a series of pulses to set a “time zero” for the datum light sensor 124. In some embodiments, the light emitted by the pulsed light emitter 600 translates or rotates with respect to the mouth of the patent. In some embodiments, the pulsed light emitter 600 translates or rotates with respect to the mouth of the patent. In some embodiments, the light emitted by the pulsed light emitter 600 scans the entire visible portion of the mouth of the patient. In some embodiments, the light emitted by the pulsed light emitter 600 scans the entire visible portion of the mouth of the patient at a scan rate such that movement of the datum 120 between scans is relatively negligible. In some embodiments, the pulsed light emitter 600 emits a predetermined series of pulses to set a “time zero” for the datum light sensor 124. In some embodiments, the datum transmission module further transmits the sensed light. In some embodiments, the probe transmission module further transmits the sensed light. In some embodiments, the orientation of the datum 120 is based on the light sensed by the light sensor. In some embodiments, the modeling system determines the second morphology based on the time at which each of the photodetectors detects the emitted light relative to its position on the datum 120 and the time of the initial flash. In some embodiments, the modeling system further determines the second morphology based an angle at which the light was emitted from the pulsed light emitter 600.

### Methods for Mapping a Three-Dimensional (3D) Dental Anatomy

Another aspect provided herein is a method for mapping a three-dimensional (3D) dental anatomy of a subject, the method comprising: capturing, by a non-contact sensor, a first morphology data of at least a portion of the mouth of the subject; transmitting, by a sensor transmission module, the first morphology data; measuring, by a probe orientation sensor having a probe contact surface, an orientation of a probe while the probe contact surface contacts the mouth of the subject; transmitting, by a probe transmission module, the orientation of the probe; determining the 3D dental anatomy based on the first morphology data and the orientation of the probe.

In some embodiments, capturing the first morphology comprises point-by-point scanning, line scanning, raster scanning, full-field scanning, or any combination thereof. In some embodiments, capturing the first morphology comprises activating a switch to initiate the capture of the first morphological data, terminate the capture of the first morphological data, or both. In some embodiments, the method further comprises combining the first morphology data from two or more non-contact sensors. In some embodiments, combining the first morphology data from two or more non-contact sensors comprises triangulation, stereophotogrammetry, or both.

In some embodiments, the first morphology data, the second morphology data, the 3D dental anatomy, or any combination thereof comprises a point cloud, a mesh, a surface model, or any combination thereof. In some embodiments, the first morphology data, the second morphology data, the 3D dental anatomy, or any combination thereof comprises a 3D surface data, a 3D volumetric data, or both. In some embodiments, the method further comprises converting the 3D surface data to the 3D volumetric data. In some embodiments, the method further comprises converting the 3D volumetric data to the 3D surface data. In some embodiments, converting the 3D volumetric data to the 3D surface data comprises segmenting and/or tessellating the 3D volume into two or more components. In some embodiments, the component comprises a gingiva component, a tooth component, a decay component, a pulp component, or any combination thereof. In some embodiments, the first morphology comprises a visible surface morphology, a subsurface occluded morphology, or both. In some embodiments, the visible surface morphology comprises a tooth surface, a gum surface, a cheek surface, a tongue surface, or any combination thereof. In some embodiments, the subsurface occluded morphology represents a morphology within a dental tissue. In some embodiments, the subsurface occluded morphology comprises a tooth pulp, a muscle, a nerve, a blood vessel, or any combination thereof.

In some embodiments, the orientation of the probe comprises a rotation of the probe about one or more axes, a translation of the probe in one or more directions, a rotational velocity of the probe about one or more axes, a translational velocity of the probe in one or more directions, an angular acceleration of the probe about one or more axes, a translational acceleration of the probe in one or more directions, or any combination thereof.

In some embodiments, the method further comprises measuring, by a datum orientation sensor, an orientation of the datum and transmitting, by a probe transmission module, the orientation of the datum. In some embodiments, the method does not comprise measuring, by a datum orientation sensor, an orientation of the datum and transmitting, by a probe transmission module, the orientation of the datum. In some embodiments, the second morphology data is determined based on the orientation of the probe and the orientation of the datum. In some embodiments, the 3D dental anatomy is determined based on the first morphology data and the second morphology data. In some embodiments, the method further comprises mounting the datum to the subject with a datum fastener. In some embodiments, mounting the datum to the subject comprises rigidly and removably mounting the datum to the subject. In some embodiments, mounting the datum to the subject comprises mounting the datum to a tooth of the subject, the jaw of the subject, or both. In some embodiments, mounting the datum to the subject comprises mounting the datum outside the subject's mouth. In some embodiments, the method further comprises capturing, by the non-contact sensor, a datum fiducial on the datum. In some embodiments, first morphology data comprises the datum fiducial. In some embodiments, the orientation of the datum comprises a rotation of the datum about one or more axes, a translation of the datum in one or more directions, a rotational velocity of the datum about one or more axes, a translational velocity of the datum in one or more directions, an angular acceleration of the datum about one or more axes, a translational acceleration of the datum in one or more directions, or any combination thereof. In some embodiments, the method further comprises extrapolating the 3D dental anatomy. In some embodiments, extrapolating the 3D dental anatomy is performed by a fourth machine learning algorithm. In some embodiments, the modeling system further interpolates the 3D dental anatomy. In some embodiments, the modeling system interpolates the 3D dental anatomy using a fifth machine learning algorithm.

In some embodiments, the confocal laser scanning microscope, the multi-photon microscope, or both, capture an image of a fluorescence within the oral cavity of the subject. In some embodiments, the method further comprises applying the fluorescence to the oral cavity of the subject. In some embodiments, the method does not comprises applying the fluorescence to the oral cavity of the subject, wherein a fluorescence of a tissue of the subject (e.g. a tooth) is measured. In some embodiments, the method further comprises mounting a reference fiducial to the subject. In some embodiments, the first morphology data further comprises a location of the reference fiducial, orientation of the reference fiducial, or both. In some embodiments, the method further comprises one or more of: measuring, by a force sensor, a probe force between the probe contact surface and a dental surface of the subject; and determining, by a touch sensor, if the distal probe contacts the dental surface of the subject. In some embodiments, the method further comprises transmitting, by the transmission module, the probe force, the contact determination, or both. In some embodiments, the 3D dental anatomy is further based on the probe force, the contact determination, or both. In some embodiments, a center axis of at least a portion of the probe contact surface is parallel to a center axis of at least a portion of the handle. In some embodiments, the probe further comprises a probe fiducial visible to the non-contact sensor. In some embodiments, first morphology data comprises the probe fiducial.

In some embodiments, the probe further comprises a probe light sensor, and wherein the method further comprises emitting a light beam by a pulsed light emitter. In some embodiments, the method further comprises translating the light beam, rotating the light beam, or both, with respect to the probe light sensor. In some embodiments, the method further comprises transmitting, by the probe transmission module, the sensed probe light. In some embodiments, 3D dental anatomy is further based on the sensed probe light. In some embodiments, the datum further comprises a datum light sensor, and wherein the method further comprises emitting a light beam by a pulsed light emitter. In some embodiments, the method further comprises translating the light beam, rotating the light beam, or both, with respect to the datum light sensor.

In some embodiments, the method further comprises, by the datum transmission module, the sensed datum light. In some embodiments, the 3D dental anatomy is further based on the sensed datum light. In some embodiments, the method further comprises orienting, by an actuator, the non-contact mapping system, the probe, or both. In some embodiments, the method further comprises capturing the first morphology at a first location, orienting, by the actuator, the non-contact sensor to a second location different from the first location, and capturing, by the non-contact sensor, a third morphology at the second location. In some embodiments, the method further comprises measuring, by an encoder, a position of the non-contact mapping system, the probe, or both. In some embodiments, the first morphology, the second morphology, or both are based on a measurement by the passive encoder. In some embodiments, the encoder is a rotational encoder. In some embodiments, the encoder is a translational encoder. In some embodiments, the method further comprises coupling the non-contact mapping sensor to the mouth of the subject. In some embodiments, the 3D dental anatomy is further based on a historical morphology data of the subject.

In some embodiments, the method further comprises normalizing the first morphology data, the second morphology data, or both. In some embodiments, the first morphology data, the second morphology data, or both are normalized by a first machine learning algorithm. In some embodiments, the 3D dental anatomy is further based on a predetermined anatomy landmark. In some embodiments, the 3D dental anatomy is determined by triangulation, confocal imaging, stereophotogrammetry, or any combination thereof. In some embodiments, the anatomy landmark comprises a gum margin, a marked tooth, or both. In some embodiments, the 3D dental anatomy is determined by applying a second machine learning algorithm. In some embodiments, the method further comprises determining an anatomy classification based on the first morphology data and the second morphology data. In some embodiments, the anatomy classification comprises a tooth classification, a gum classification, a lip classification, a cheek classification, a tongue classification, or any combination thereof. In some embodiments, the anatomy classification is determined by applying a third machine learning algorithm. In some embodiments, the method further comprises performing a dental surgery based at least in part on the 3D dental anatomy of the subject. In some embodiments, the dental anatomy is of a tooth, a jaw, a gum, a lingual tooth surface, a subgingival surface, an interproximal gap, or any combination thereof.

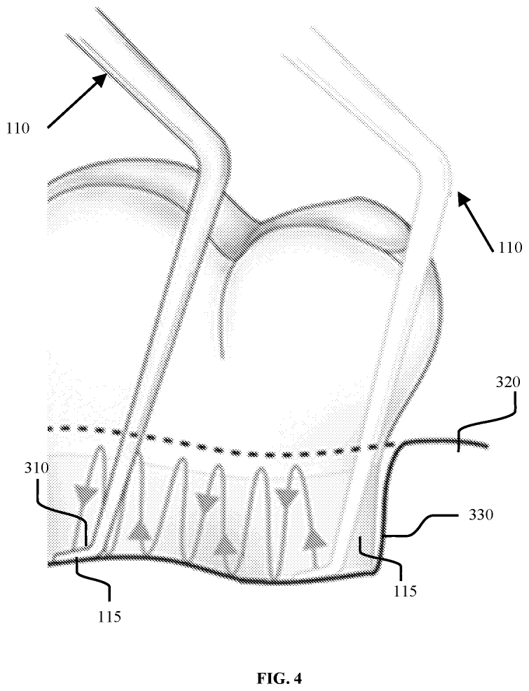

FIG. 4 shows an exemplary illustration of contacting the mouth of the subject by the probe contact surface 115. In some embodiments, contacting the mouth of the subject by the probe contact surface 115 comprises placing the probe contact surface 115 of the probe 110 within a periodontal pocket 330 within the gingiva 320 of the subject and maintaining contact between the probe contact surface 115 and the tooth of the subject while traversing the probe contact surface 115 through an exploring path 310.

### Non-Contact Sensor

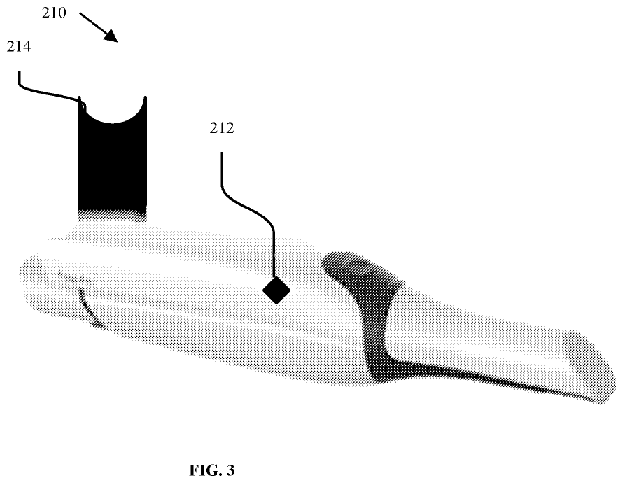

An illustration of an exemplary non-contact sensor 210 is shown in FIG. 3. In some embodiments, the non-contact sensor 210 captures a first morphology data of at least a portion of the mouth of the subject. In some embodiments, the non-contact sensor 210 comprises a 3D scanner, a LIDAR, a RADAR, a laser, a camera, a microscope, an optical coherence tomogram (OCT), a confocal laser scanning microscope (CLSM), or any combination thereof. In some embodiments, the OCT comprises a time-domain OCT, a Fourier-domain OCT, a swept-source OCT, or any combination thereof. In some embodiments, the camera comprises an endoscopic camera. In some embodiments, the endoscopic camera comprises an illumination source. In some embodiments, the microscope comprises a confocal laser scanning microscope, a multi-photon microscope, or both. In some embodiments, the confocal laser scanning microscope, the multi-photon microscope, or both captures an image of the oral cavity of the subject and a fluorescence within the oral cavity of the subject.

In some embodiments, the non-contact sensor 210 captures the first morphology by point-by-point scanning, line scanning, raster scanning, full-field scanning, or any combination thereof. In some embodiments, the non-contact mapping system further comprises a switch to initiate the capture of the first morphological data, terminate the capture of the first morphological data, or both. In some embodiments, the switch is integrated into the non-contact sensor 210.

In some embodiments, the non-contact mapping system further comprises a reference fiducial rigidly and removably mountable to the subject. In some embodiments, the reference fiducial rigidly and removably mounts to the subject by a clip, an adhesive, a clamp, a band, a tie, or any combination thereof. In some embodiments, the reference fiducial rigidly and removably mounts to one or more teeth of the subject by a clip, an adhesive, a clamp, a band, a tie, or any combination thereof. In some embodiments, the reference fiducial is a visual indicator of size and or orientation with respect to one or more teeth to which the reference fiducial is mounted to. In some embodiments, the non-contact sensor 210 further captures the reference fiducial, wherein the first morphology data further comprises a location of the reference fiducial, orientation of the reference fiducial, or both.

In some embodiments, the non-contact sensor 210 comprises a second coupling 214 that removably connects to the actuator. In some embodiments, the second coupling 214 comprises a threaded feature, a clamp, a pin, a screw, a magnet, a clamp, or any combination thereof.

In some embodiments, the non-contact sensor 210 further comprises a non-contact sensor fastener. In some embodiments, the non-contact sensor fastener mounts the non-contact sensor 210 to the subject. In some embodiments, the non-contact sensor fastener mounts the non-contact sensor 210 to the head of the subject. In some embodiments, the non-contact sensor fastener comprises a clip, an adhesive, a clamp, a band, a tie, or any combination thereof. In some embodiments, the non-contact sensor fastener rigidly and removably mounts to the subject. In some embodiments, the non-contact sensor fastener mounts to a tooth of the subject, the jaw of the subject, or both. In some embodiments, the non-contact sensor fastener reduces and/or eliminates any mapping errors formed by relative motion between the non-contact sensor 210 and the head of the patient.

In some embodiments, the sensor transmission module transmits the first morphology data. In some embodiments, the sensor transmission module comprises a Bluetooth transmitter, a Wi-Fi transmitter, a cellular transmitter, a wired transmitter, an optical transmitter, or any combination thereof.

### Probe

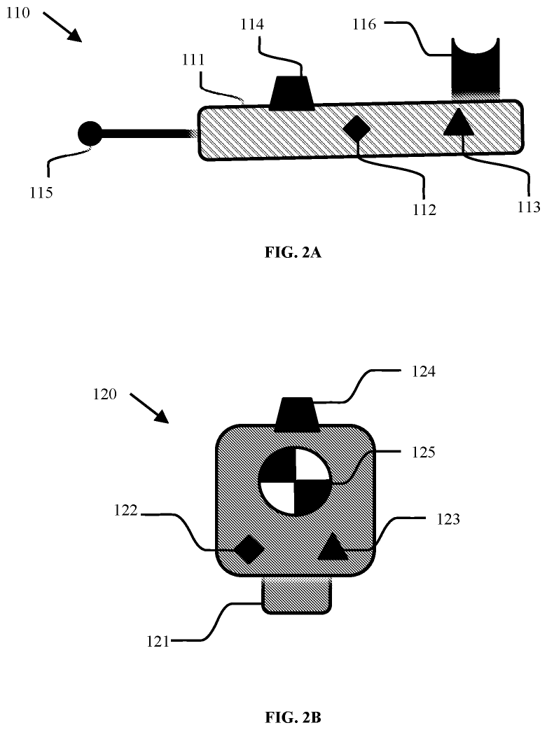

FIG. 2A provides an illustration of an exemplary probe 110. In some embodiments, the probe 110 comprises a periodontal endoscope. In some embodiments, the probe 110 is a sub-gingival probe 110. In some embodiments, the probe 110 comprises a probe orientation sensor 113 measuring an orientation of the probe 110, a probe contact surface 115, and a probe transmission module 112 transmitting the orientation of the probe 110. In some embodiments, the probe orientation sensor 113 measures an orientation of the probe 110. In some embodiments, the probe orientation sensor 113 comprises an accelerometer, a tilt sensor, a gyroscope, a GPS sensor, a distance sensor, a RADAR, a magnet, a radio frequency generator, a radio frequency receiver, or any combination thereof. In some embodiments, the probe orientation sensor 113 is located within at most about 2 inches from the center of mass of the probe 110. In some embodiments, the orientation of the probe 110 comprises a rotation of the probe 110 about one or more axes, a translation of the probe 110 in one or more directions, a rotational velocity of the probe 110 about one or more axes, a translational velocity of the probe 110 in one or more directions, an angular acceleration of the probe 110 about one or more axes, a translational acceleration of the probe 110 in one or more directions, or any combination thereof.

In some embodiments, the probe contact surface 115 comprises a periodontal endoscope. In some embodiments, the probe contact surface 115 is a sub-gingival probe 110. In some embodiments, the probe contact surface 115 is generally acute. In some embodiments, the probe contact surface 115 is rounded. In some embodiments, at least a portion of the probe contact surface 115 is rigid. In some embodiments, at least a portion of the probe contact surface 115 is flexible. In some embodiments, at least a portion of the probe contact surface 115 is removable from the probe 110. In some embodiments, the probe 110 further comprises a handle 111 coupled to the probe contact surface 115. In some embodiments, a center axis of at least a portion of the probe contact surface 115 is parallel to a center axis of at least a portion of the handle 111. In some embodiments, a center axis of at least a portion of the probe contact surface 115 is parallel to a center axis of at least a portion of the handle 111. In some embodiments, a center axis of at least a portion of the probe contact surface 115 is parallel to a center axis of at least a portion of the handle 111. In some embodiments, a distance between the probe orientation sensor 113 and a distal point of the probe contact surface 115 is constant.

In some embodiments, the probe 110 further comprises a force sensor measuring a probe force between the probe contact surface 115 and a dental surface of the subject. In some embodiments, the probe 110 further comprises a touch sensor determining if the distal probe 110 contacts the dental surface of the subject. In some embodiments, the probe 110 further comprises a probe 110 fiducial visible to the non-contact sensor. In some embodiments, first morphology data comprises the probe 110 fiducial.

In some embodiments, the probe 110 further comprises a probe light sensor 114, and wherein the platform further comprises a pulsed light emitter. In some embodiments, a beam of light emitted by the pulsed light emitter translates, rotates, or both, with respect to the probe light sensor 114. In some embodiments, the probe light sensor 114 comprises a photodiode, a photodetector, or both. In some embodiments, the pulsed light emitter is a laser, a light emitting diode (LED), or both. In some embodiments, the light emitted by the pulsed light emitter translates or rotates with respect to the mouth of the patent.

In some embodiments, the probe 110 comprises a first coupling 116 that removably connects to an actuator. In some embodiments, the first coupling 116 comprises a threaded feature, a clamp, a pin, a screw, a magnet, a clamp, or any combination thereof.

In some embodiments, the probe transmission module 112 transmits the orientation of the probe 110. In some embodiments, the probe transmission module 112 further transmits the probe force, the contact determination, or both. In some embodiments, the probe transmission module 112 further transmits the sensed probe 110 light. In some embodiments, the probe transmission module 112 further transmits a data based on the touch sensor determining if the distal probe 110 contacts the dental surface of the subject. In some embodiments, the probe transmission module 112 comprises a Bluetooth transmitter, a Wi-Fi transmitter, a cellular transmitter, a wired transmitter, an optical transmitter, or any combination thereof.

### Datum

In some embodiments, the datum orientation sensor 123 measures an orientation of the datum 120. In some embodiments, per FIG. 2B, the datum orientation sensor 123 comprises an accelerometer, a tilt sensor, a gyroscope, a GPS sensor, a distance sensor, a RADAR, a magnet, a radio frequency generator, a radio frequency receiver, or any combination thereof. In some embodiments, the orientation of the datum 120 comprises a rotation of the datum 120 about one or more axes, a translation of the datum 120 in one or more directions, a rotational velocity of the datum 120 about one or more axes, a translational velocity of the datum 120 in one or more directions, an angular acceleration of the datum 120 about one or more axes, a translational acceleration of the datum 120 in one or more directions, or any combination thereof. In some embodiments, the datum orientation sensor 123 is located within at most about 2 inches from the center of mass of the datum 120.

In some embodiments, the datum fastener 121 mounts the datum 120 to the subject. In some embodiments, the datum fastener 121 comprises a clip, an adhesive, a clamp, a band, a tie, or any combination thereof. In some embodiments, the datum fastener 121 rigidly and removably mounts to the subject. In some embodiments, the datum fastener 121 mounts to a tooth of the subject, the jaw of the subject, or both. In some embodiments, the datum fastener 121 mounts outside the subject's mouth. In some embodiments, the datum 120 further comprises a datum 120 fiducial 125 visible to the non-contact sensor. In some embodiments, first morphology data comprises the datum 120 fiducial 125.

In some embodiments, the datum 120 further comprises a datum light sensor 124, and wherein the platform further comprises a pulsed light emitter. In some embodiments, a beam of light emitted by the pulsed light emitter translates, rotates, or both, with respect to the datum light sensor 124. In some embodiments, the datum light sensor 124 comprises a photodiode, a photodetector, or both. In some embodiments, the pulsed light emitter is a laser, a light emitting diode (LED), or both. In some embodiments, the light emitted by the pulsed light emitter translates or rotates with respect to the mouth of the patent.

In some embodiments, the datum transmission module 122 transmits the orientation of the datum 120. In some embodiments, the datum transmission module 122 further transmits the sensed probe light. In some embodiments, the datum transmission module 122 comprises a Bluetooth transmitter, a Wi-Fi transmitter, a cellular transmitter, a wired transmitter, an optical transmitter, or any combination thereof.

### Modeling System

In some embodiments, the modeling system receives the first morphology data, the orientation of the probe, and the orientation of the datum. In some embodiments, the modeling system determines a second morphology data based on the orientation of the probe. In some embodiments, the modeling system determines the second morphology data based on the orientation of the probe and the orientation of the datum. In some embodiments, the modeling system further determines the second morphology data based on a distance between the probe orientation sensor and a distal tip of the probe contact surface. In some embodiments, the modeling system determines the second morphology based on the time at which photodetectors on the probe, the datum, or both detect emitted light relative to its position on the probe and the time of the initial flash. In some embodiments, the modeling system further determines the second morphology based an angle at which the light was emitted from the pulsed light emitter.

In some embodiments, the modeling system determines the 3D dental anatomy based on the first morphology data. In some embodiments, the modeling system determines the 3D dental anatomy based on the first morphology data and the second morphology data. In some embodiments, the first morphology data, the second morphology data, or both comprise a one-dimensional morphology data, a two-dimensional morphology data, or a three-dimensional morphology data. In some embodiments, the 3D dental anatomy is formed by combining a plurality of one-dimensional morphology data, two-dimensional morphology data, three-dimensional morphology data, or any combination thereof

In some embodiments, the modeling system determines the 3D dental anatomy by combining the first morphology data and the second morphology data. In some embodiments, the modeling system determines the 3D dental anatomy by combining a plurality of one-dimensional morphology data, two-dimensional morphology data, three-dimensional morphology data, or any combination thereof. In some embodiments, the combination comprises fitting overlapping surfaces. In some embodiments, the combination comprises fitting overlapping surfaces based on a location of the non-contact sensor, the contact sensor, or both. In some embodiments, the combination comprises fitting overlapping surfaces without requiring a location of the non-contact sensor, the contact sensor, or both.

In some embodiments, the modeling system further determines the 3D dental anatomy based on the probe force, the contact determination, or both. In some embodiments, the modeling system further determines the 3D dental anatomy based on the sensed probe light. In some embodiments, the modeling system further determines the 3D dental anatomy based on the sensed datum light. In some embodiments, the modeling system further normalizes the first morphology data based on a translation and/or rotation of a location of the reference fiducial, orientation of the reference fiducial, or both mounted to a tooth of the subject.

In some embodiments, the modeling system further determines the 3D dental anatomy based on a historical morphology data of the subject. In some embodiments, the modeling system normalizes the first morphology data, the second morphology data, or both. In some embodiments, the first morphology data, the second morphology data, or both are normalized by a first machine learning algorithm. In some embodiments, the modeling system further determines the 3D dental anatomy based on a predetermined anatomy landmark. In some embodiments, the modeling system determines the 3D dental anatomy by triangulation, confocal imaging, stereophotogrammetry, or any combination thereof. In some embodiments, the anatomy landmark comprises a gum margin, a marked tooth, or both. In some embodiments, the modeling system further determines the 3D dental anatomy by applying a second machine learning algorithm.

In some embodiments, the modeling system determines the second morphology based on the time at which each of the photodetectors detects the emitted light relative to its position on the probe and the time of the initial flash. In some embodiments, the modeling system further determines the second morphology based an angle at which the light was emitted from the pulsed light emitter.

In some embodiments, the modeling system further determines an anatomy classification based on the first morphology data and the second morphology data. In some embodiments, the modeling system further determines the anatomy classification based on the probe force. In some embodiments, the anatomy classification comprises a tooth classification, a gum classification, a lip classification, a cheek classification, a tongue classification, or any combination thereof. In some embodiments, the modeling system determines the anatomy classification by applying a third machine learning algorithm. In some embodiments, the modeling system further extrapolates the 3D dental anatomy. In some embodiments, the modeling system extrapolates the 3D dental anatomy using a fourth machine learning algorithm. In some embodiments, extrapolating the 3D dental anatomy comprises a normal single-axis extrapolation of an edge, a normal linear gradient extrapolation of the edge surfaces, determining a polynomial gradient projection of a surface adjacent to an edge, or any combination thereof. In some embodiments, the modeling system further interpolates the 3D dental anatomy. In some embodiments, the modeling system interpolates the 3D dental anatomy using a fifth machine learning algorithm.

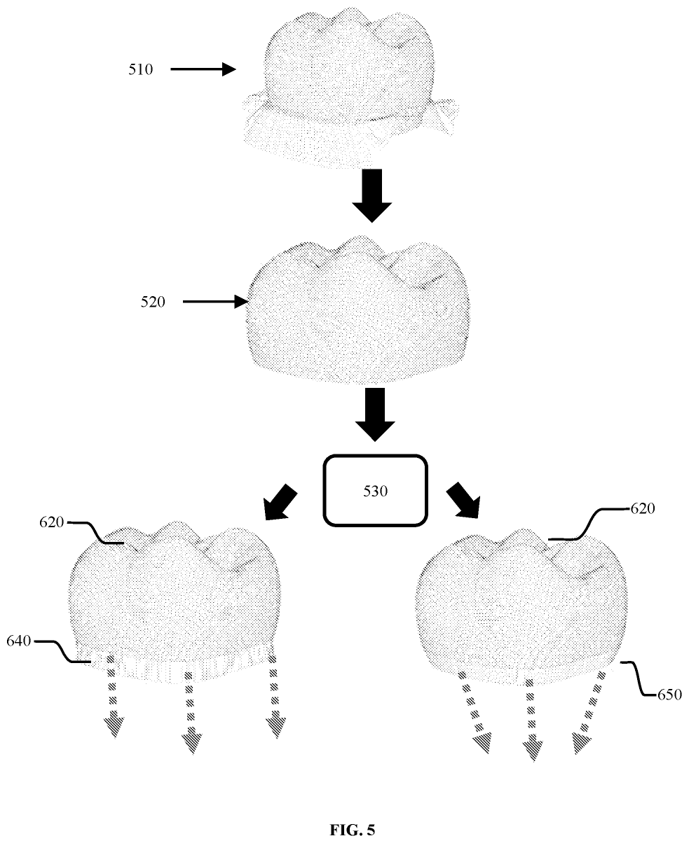

FIG. 5 shows a diagram of an exemplary method of determining a polynomial gradient projection, wherein a 3D dental anatomy 610 captured by the contact sensor, the non-contact sensor, or both is segmented to form a segmented 3D dental anatomy 620. In some embodiments, based on a received input 630, a vertical projection 640 is formed from segmented 3D dental anatomy 620, or a gradient projection 650 is formed from the segmented 3D dental anatomy 620.

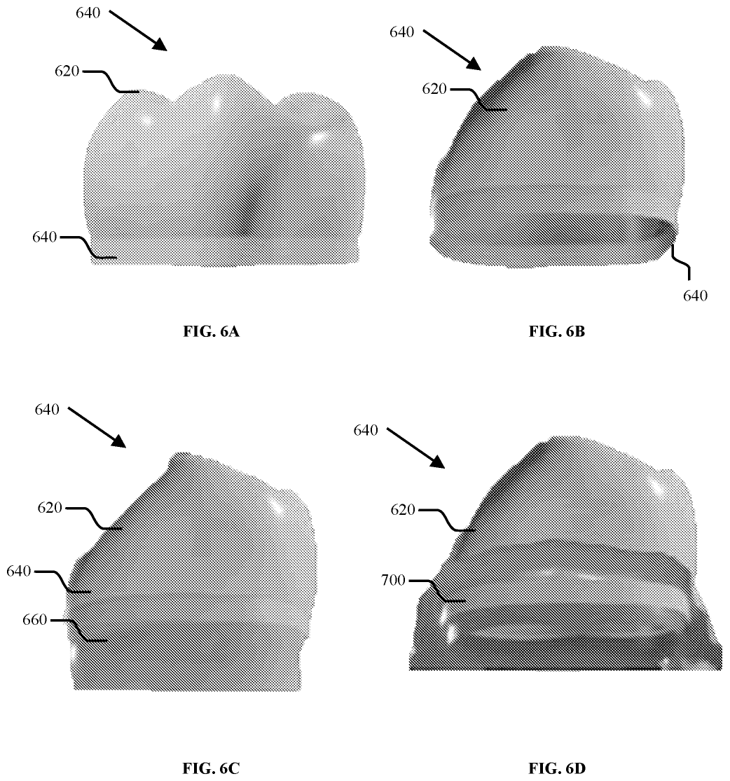

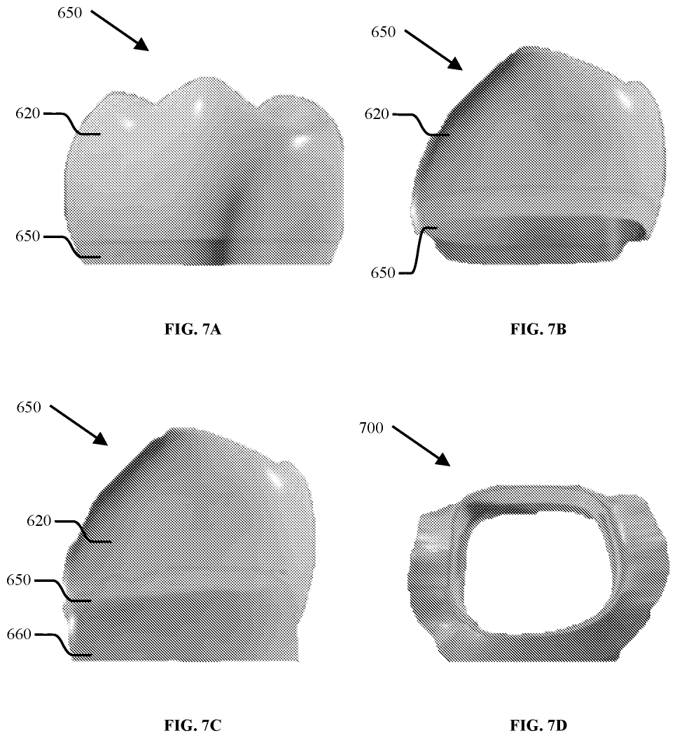

FIGS. 6A-B show a front view and a front-bottom perspective view, respectively, of an exemplary segmented 3D dental anatomy 620 and a vertical projection 640 extending therefrom. FIG. 6C shows a front view image of the exemplary segmented 3D dental anatomy 620, the vertical projection 640, and a model of an existing tooth 660. FIG. 6D shows a front-bottom perspective view image of the exemplary segmented 3D dental anatomy 620, the vertical projection 640 and an exemplary gum surface 700. FIGS. 7A-B show a front view and a front-bottom perspective view, respectively, of an exemplary segmented 3D dental anatomy 620 and a gradient projection 650 extending therefrom. FIG. 7C shows a front view image of the exemplary segmented 3D dental anatomy 620, the gradient projection 650, and a model of an existing tooth 660. FIG. 7D shows a front-bottom perspective view image of an exemplary gum surface 700.

### Assisted Operation

In some embodiments, the platform further comprises an actuator coupled to the non-contact sensor, the probe, or both. In some embodiments, the platform comprises a plurality of actuators. In some embodiments, the platform comprises 2, 3, 4, 5, 6, 7, 8, 9, 10, 11, 12 or more actuators. In some embodiments, the platform comprises 2, 3, 4, 5, 6, 7, 8, 9, 10, 11, 12 or more actuators, wherein each actuator translates or rotates the non-contact sensor, the probe, or both. In some embodiments, two or more of the plurality of actuators translate the non-contact sensor, the probe, or both in orthogonal directions. In some embodiments, two or more of the plurality of actuators rotate the non-contact sensor, the probe, or both about orthogonal axis.

In some embodiments, the non-contact sensor captures a first morphology at a first location, wherein the actuator orients the non-contact sensor to a second location different from the first location, and wherein the non-contact sensor captures a third morphology at the second location. In some embodiments, the non-contact sensor captures a first morphology at a first rotational position, wherein the actuator orients the non-contact sensor to a second rotational position different from the first rotational position, and wherein the non-contact sensor captures a third morphology at the second location.

In some embodiments, the actuator comprises the actuators and/or systems as disclosed in any one of PCT Publication Nos. WO2017130060, WO2018154485, or WO2019215512A1. In some embodiments, the actuator comprises a robotic arm. In some embodiments, the actuator is incorporated into a robotic arm.

In some embodiments, the platform further comprises a encoder measuring a position of the non-contact mapping system, the probe, or both. In some embodiments, the platform further comprises a encoder measuring an orientation of the non-contact mapping system, the probe, or both. In some embodiments, the first morphology, the second morphology, or both are based on a measurement by the passive encoder. In some embodiments, the encoder is a rotational encoder. In some embodiments, the encoder is a translational encoder. In some embodiments, the platform further comprises a mouth coupling device removably coupling the non-contact mapping to the mouth of the patient. In some embodiments, the mouth coupling device comprises a clamp, a threaded feature, a band, an adhesive, or any combination thereof.

### Optical Coherence Tomography

Optical coherence tomography (OCT) is a technique for capturing 3D volumetric images of occluded structures. In some embodiments, OCT records such images by measuring a depth-resolved reflectivity profile caused by optical interference between a reference light beam and light reflected from within an object at a set depth. In some embodiments, OCT records such images by measuring a depth-resolved reflectivity profile caused by optical interference between a reference light beam and light reflected from within an object at all tissue depths. In some embodiments, the OCT captures the 3D depth-resolved volumetric images by point-by-point scanning, line scanning, raster scanning, full-field scanning, or any combination thereof. In some embodiments, the OCT comprises a swept-source OCT sensor, a time-domain OCT sensor, a spectral domain OCT sensor, or any combination thereof

In some embodiments, point-by-point scanning comprises focusing a single point light at a fixed location relative to the OCT sensor (e.g. incident on the sample). In some embodiments, point-by-point scanning enables measurement by the OCT of a depth reflectivity profile below the single point. In some embodiments, the first morphology data of at least a portion of the mouth of the subject is formed by combining OCT measurements at various depths and fixed points.

In some embodiments, line scanning comprises translating the OCT sensor's point of focus along a first axis to measure a 2D depth-resolved reflectivity cross section. In some embodiments, the OCT sensor's point of focus is translated by reflecting the OCT's beam in an oscillating mirror. In some embodiments, the OCT is translated in a direction approximately perpendicular to the first axis, wherein the first morphology data of at least a portion of the mouth of the subject is formed by combining two or more 2D depth-resolved reflectivity cross sections.

In some embodiments, raster scanning comprises translating the OCT sensor's point of focus along two axes to measure the first morphology data of at least a portion of the mouth of the subject. In some embodiments, the OCT sensor's point of focus is translated by reflecting the OCT's beam in two oscillating mirrors. In some embodiments, the first morphology data of at least a portion of the mouth of the subject is formed by combining a plurality of raster scans, each raster scan at a different depth. In some embodiments, full-field scanning comprises simultaneous raster scanning by a plurality of OCT sensors, each sensor focused at a different depth.

### Confocal Laser Scanning Microscopy

In some embodiments, confocal laser scanning microscopy (CLSM) captures a 3D volumetric image of a sample. In some embodiments, CLSM employs a microscope and an optical detection scheme blocking all light that does not emerge from the microscope's focal plane. In some embodiments, the optical detection scheme blocks all light at a certain depth relative to the microscope's objective/scanning lens. In some embodiments, CSLM delivers and/or collects light through a fiber or fiber bundle. In some embodiments, CLSM is a point-scanning method (i.e. it images a single point in 3D space at a time). In some embodiments, depth scanning with the CLSM comprises moving the focal plane of the CLSM sensor. In some embodiments, the focal plane is moved by translating the CLSM sensor, its beam, or both. In some embodiments, transverse scanning is performed

In some embodiments, the 3D depth-resolved volumetric image is captured by point-by-point scanning, line scanning, raster scanning, full-field scanning, or any combination thereof, whereas the combination of data recorded in one or more direction is compiled.

In some embodiments, confocal laser scanning microscope comprises fluorescence confocal laser scanning microscope having an excitation light. In some embodiments, the excitation light is a laser. In some embodiments, the confocal laser scanning microscope isolates the emitted fluorescence to provide providing axial and/or depth resolution. In some embodiments, the excitation light and any non-fluoresced light is blocked from the CLSM by an optical filter. In some embodiments, the excitation light and any non-fluoresced light is not blocked from the CLSM by an optical filter. In some embodiments, the fluorescence is a natural fluorescence of excited teeth and/or gums. In some embodiments, the fluorescence is emitted from applied fluorophore. In some embodiments, the fluorophore is applied topically, intravenously, or both.

### Multiphoton Microscopy

In some embodiments, multi-photon microscopy comprises stimulating a fluorescence by a high intensity photon beam. In some embodiments, multi-photon microscopy comprises stimulating a fluorescence at the intersection of two or more photon beams. In some embodiments, the strong excitation provided by such an intersection enables imaging with higher resolution. In some embodiments, the excitation light is a laser. In some embodiments, the excitation wavelength is longer than the emission wavelength, wherein the excitation light and any non-fluoresced light is blocked with an optical filter. In some embodiments, a series of two longer wavelength photons are absorbed and a single shorter wavelength photon is emitted upon fluorescence. In some embodiments, the excitation light and any non-fluoresced light is not blocked with an optical filter. In some embodiments, multi-photon microscopy is inherently confocal, wherein out-of-focus light is rejected without the requisite for additional filtering optics, which thus further improves the imaging resolution. In some embodiments, multi-photon microscopy enables imaging of deeper tissue, efficient light detection, and reduced photobleaching. In some embodiments, the fluorescence is a natural fluorescence of excited teeth and/or gums. In some embodiments, the fluorescence is emitted from applied fluorophore. In some embodiments, the fluorophore is applied topically, intravenously, or both.

### Focus Stacking

In some embodiments, focus stacking comprises adjusting the focus of a camera (i.e. microscope) to capture images over a range of tissue depths. In some embodiments, adjusting the focus of a camera comprises adjusting a focus length of the camera. In some embodiments, identification of the in-focus regions implicitly identifies the depth of the surface being imaged, wherein the resultant depth profiles are stitched together. In some embodiments, focus stacking comprises occluding scattered/reflected light. In some embodiments, focus stacking comprises suppressing scattered/reflected light, by an optical component, for example, a polarizer, or by software-based post-processing. In some embodiments, the wavelength of the light projected onto and into the dental tissue is selected to reduce such scattered/reflected light.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

### Computing System

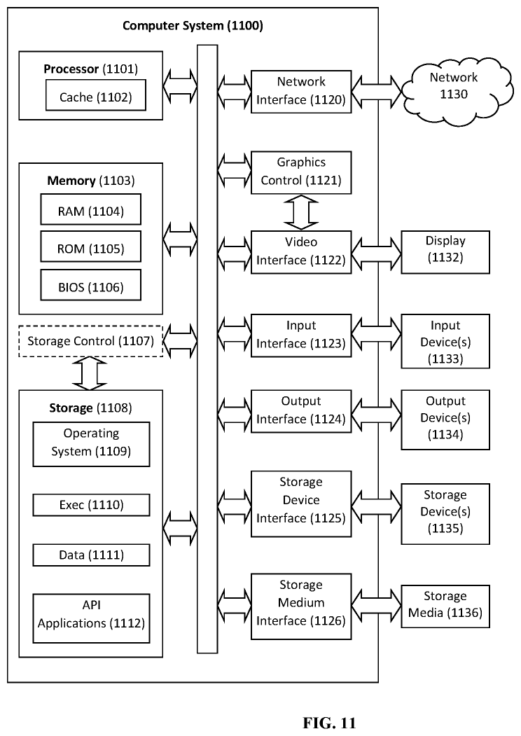

Referring to FIG. 11, a block diagram is shown depicting an exemplary machine that includes a computer system 1100 (e.g., a processing or computing system) within which a set of instructions can execute for causing a device to perform or execute any one or more of the aspects and/or methodologies for static code scheduling of the present disclosure. The components in FIG. 11 are examples only and do not limit the scope of use or functionality of any hardware, software, embedded logic component, or a combination of two or more such components implementing particular embodiments.

Computer system 1100 may include one or more processors 1101, a memory 1103, and a storage 1108 that communicate with each other, and with other components, via a bus 1140. The bus 1140 may also link a display 1132, one or more input devices 1133 (which may, for example, include a keypad, a keyboard, a mouse, a stylus, etc.), one or more output devices 1134, one or more storage devices 1135, and various tangible storage media 1136. All of these elements may interface directly or via one or more interfaces or adaptors to the bus 1140. For instance, the various tangible storage media 1136 can interface with the bus 1140 via storage medium interface 1126. Computer system 1100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 1100 includes one or more processor(s) 1101 (e.g., central processing units (CPUs) or general purpose graphics processing units (GPGPUs)) that carry out functions. Processor(s) 1101 optionally contains a cache memory unit 1102 for temporary local storage of instructions, data, or computer addresses. Processor(s) 1101 are configured to assist in execution of computer readable instructions. Computer system 1100 may provide functionality for the components depicted in FIG. 11 as a result of the processor(s) 1101 executing non-transitory, processor-executable instructions embodied in one or more tangible computer-readable storage media, such as memory 1103, storage 1108, storage devices 1135, and/or storage medium 1136. The computer-readable media may store software that implements particular embodiments, and processor(s) 1101 may execute the software. Memory 1103 may read the software from one or more other computer-readable media (such as mass storage device(s) 1135, 1136) or from one or more other sources through a suitable interface, such as network interface 1120. The software may cause processor(s) 1101 to carry out one or more processes or one or more steps of one or more processes described or illustrated herein. Carrying out such processes or steps may include defining data structures stored in memory 1103 and modifying the data structures as directed by the software.

The memory 1103 may include various components (e.g., machine readable media) including, but not limited to, a random access memory component (e.g., RAM 1104) (e.g., static RAM (SRAM), dynamic RAM (DRAM), ferroelectric random access memory (FRAM), phase-change random access memory (PRAM), etc.), a read-only memory component (e.g., ROM 1105), and any combinations thereof. ROM 1105 may act to communicate data and instructions unidirectionally to processor(s) 1101, and RAM 1104 may act to communicate data and instructions bidirectionally with processor(s) 1101. ROM 1105 and RAM 1104 may include any suitable tangible computer-readable media described below. In one example, a basic input/output system 1106 (BIOS), including basic routines that help to transfer information between elements within computer system 1100, such as during start-up, may be stored in the memory 1103.

Fixed storage 1108 is connected bidirectionally to processor(s) 1101, optionally through storage control unit 1107. Fixed storage 1108 provides additional data storage capacity and may also include any suitable tangible computer-readable media described herein. Storage 1108 may be used to store operating system 1109, executable(s) 1110, data 1111, applications 1112 (application programs), and the like. Storage 1108 can also include an optical disk drive, a solid-state memory device (e.g., flash-based systems), or a combination of any of the above. Information in storage 1108 may, in appropriate cases, be incorporated as virtual memory in memory 1103.

In one example, storage device(s) 1135 may be removably interfaced with computer system 1100 (e.g., via an external port connector (not shown)) via a storage device interface 1125. Particularly, storage device(s) 1135 and an associated machine-readable medium may provide non-volatile and/or volatile storage of machine-readable instructions, data structures, program modules, and/or other data for the computer system 1100. In one example, software may reside, completely or partially, within a machine-readable medium on storage device(s) 1135. In another example, software may reside, completely or partially, within processor(s) 1101.

Bus 1140 connects a wide variety of subsystems. Herein, reference to a bus may encompass one or more digital signal lines serving a common function, where appropriate. Bus 1140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 1100 may also include an input device 1133. In one example, a user of computer system 1100 may enter commands and/or other information into computer system 1100 via input device(s) 1133. Examples of an input device(s) 1133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 1133 may be interfaced to bus 1140 via any of a variety of input interfaces 1123 (e.g., input interface 1123) including, but not limited to, serial, parallel, game port, USB, FIREWIRE, THUNDERBOLT, or any combination of the above.

In particular embodiments, when computer system 1100 is connected to network 1130, computer system 1100 may communicate with other devices, specifically mobile devices and enterprise systems, distributed computing systems, cloud storage systems, cloud computing systems, and the like, connected to network 1130. Communications to and from computer system 1100 may be sent through network interface 1120. For example, network interface 1120 may receive incoming communications (such as requests or responses from other devices) in the form of one or more packets (such as Internet Protocol (IP) packets) from network 1130, and computer system 1100 may store the incoming communications in memory 1103 for processing. Computer system 1100 may similarly store outgoing communications (such as requests or responses to other devices) in the form of one or more packets in memory 1103 and communicated to network 1130 from network interface 1120. Processor(s) 1101 may access these communication packets stored in memory 1103 for processing.

Examples of the network interface 1120 include, but are not limited to, a network interface card, a modem, and any combination thereof. Examples of a network 1130 or network segment 1130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 1130, may employ a wired and/or a wireless mode of communication. In general, any network topology may be used.

Information and data can be displayed through a display 1132. Examples of a display 1132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 1132 can interface to the processor(s) 1101, memory 1103, and fixed storage 1108, as well as other devices, such as input device(s) 1133, via the bus 1140. The display 1132 is linked to the bus 1140 via a video interface 1122, and transport of data between the display 1132 and the bus 1140 can be controlled via the graphics control 1121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 1132, computer system 1100 may include one or more other peripheral output devices 1134 including, but not limited to, an audio speaker, a printer, a storage device, and any combinations thereof. Such peripheral output devices may be connected to the bus 1140 via an output interface 1124. Examples of an output interface 1124 include, but are not limited to, a serial port, a parallel connection, a USB port, a FIREWIRE port, a THUNDERBOLT port, and any combinations thereof.

In addition or as an alternative, computer system 1100 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of dental morphology data. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

### Machine Learning

In some embodiments, machine learning algorithms are utilized to aid in normalizing the first morphology data, the second morphology data, or both. In some embodiments, machine learning algorithms are utilized to determine a 3D dental anatomy based on a predetermined anatomy landmark. In some embodiments, machine learning algorithms are utilized to determine the anatomy classification. In some embodiments, machine learning algorithms are utilized to extrapolate a 3D surface. In some embodiments, machine learning algorithms are utilized to interpolate a 3D surface.

In some embodiments, the machine learning algorithms utilized by the modeling system employ one or more forms of labels including but not limited to human annotated labels and semi-supervised labels. The human annotated labels can be provided by a hand-crafted heuristic. The semi-supervised labels can be determined using a clustering technique to find properties similar to those flagged by previous human annotated labels and previous semi-supervised labels. The semi-supervised labels can employ a XGBoost, a neural network, or both.

In some embodiments, the modeling system normalizes the first morphology data, the second morphology data, or both using a distant supervision method. In some embodiments, the modeling system determines a 3D dental anatomy based on a predetermined anatomy landmark using a distant supervision method. The distant supervision method can create a large training set seeded by a small hand-annotated training set. The distant supervision method can comprise positive-unlabeled learning with the training set as the ‘positive’ class. The distant supervision method can employ a logistic regression model, a recurrent neural network, or both. The recurrent neural network can be advantageous for Natural Language Processing (NLP) machine learning.

Examples of machine learning algorithms can include a support vector machine (SVM), a naive Bayes classification, a random forest, a neural network, deep learning, or other supervised learning algorithm or unsupervised learning algorithm for classification and regression. The machine learning algorithms can be trained using one or more training datasets.

A non-limiting example of a multi-variate linear regression model algorithm is seen below: probability 32 A₀+A₁(X₁)+A₂(X₂)+A₃(X₃)+A₄(X₄)+A₅(X₅)+A₆(X₆)+A₇(X₇) . . . wherein A₁ (A₁, A₂, A₃, A₄, A₅, A₆, A₇, . . . ) are “weights” or coefficients found during the regression modeling; and X₁ (X₁, X₂, X₃, X₄, X₅, X₆, X₇, . . . ) are data collected from the User. Any number of A₁ and X₁ variable can be included in the model. In some embodiments, the programming language “R” is used to run the model.

In some embodiments, training comprises multiple steps. In a first step, an initial model is constructed by assigning probability weights to predictor variables. In a second step, the initial model is used to “recommend” an initial morphology. In a third step, the validation module accepts verified data regarding an alternatively measured morphology and feeds back the verified data to the modeling system. At least one of the first step, the second step, and the third step can repeat one or more times continuously or at set intervals.

### Additional Platforms and Methods

Optical coherence tomography (OCT) is a technique with the ability to create 3D volumetric images of structures that are occluded from direct viewing. To create these 3D images, optical interference between a reference light beam and light that is reflected from depths within an object of interest is used. This interference enables the reflectivity at different depths within the sample to be resolved. By measuring the depth-resolved reflectivity profile at many transverse locations (either by scanning a point of light over the area of interest, or by imaging the interfered light with a camera), a 3D volumetric image of an object can be created (which includes 3D surface information).

Provided herein is a method is of imaging a portion of the 3D surface of a tooth, including various occluded regions between teeth and below the gumline. A number of studies have shown that OCT can be used to image occluded volumes within the teeth and gums. To image the full required region of the tooth surface using OCT, it is necessary to have a method of capturing multiple views of the tooth and stitching them together. Also provided herein is a platform comprising an OCT probe mounted on a robotic system used for changing the point of view of the OCT, and methods for stitching together multiple 3D OCT views using the known position and orientation of the robot.

In some embodiments, the proposed device comprises a macro positioning system robot arm with an OCT probe coupled to its distal end. In some embodiments, the robot is capable of motions along one or more degrees of freedom and would track its own relative location/orientation as it moves (by using encoders, motor step counting, or another method).

In some embodiments, the methods provided herein measure OCT volumes from multiple points of view by moving the robot and stitching the views together (using the known robot positions/orientations from which the OCT volumes were collected, image registration, or a combination thereof) to make a complete 3D image of the desired volume, from which 3D surface data can be extracted.

The OCT probe may, in some embodiments, be implemented using any of the known OCT modalities (time-domain OCT, Fourier-domain OCT, swept-source OCT, etc.). In some embodiments, the probing light for OCT can be coupled into an optical fiber which allows for simple and flexible delivery to a site of interest. To obtain 3D volumetric images, scanning over the volumes of interest may be done in multiple ways:
Movable fixed point: A single point of focused light at a location fixed relative to the OCT probe could be incident on the sample, which would enable the measurement of a depth reflectivity profile below that point. The volume of interest would be mapped by moving the probe to many adjacent locations/orientations and the full volume of interest would be collected by combining the measured adjacent depth profiles.
Line scanning: A point of focused light from the OCT probe incident on the sample could be rapidly scanned along one axis (by using one or more oscillating mirrors in the probe head or by another method) with a depth profile measured at each point along the line (line scanning). This would enable the measurement of a 2D depth-resolved reflectivity “slice”. Adjacent slices would be mapped by moving the probe to adjacent locations/orientations, which would then be combined to map the full volume of interest.
Raster scanning: A point of focused light from the OCT probe could be rapidly scanned along two axes (by using one or more oscillating mirrors in the probe head or another method) with a depth profile collected at each point in the sampled area (raster scanning). This would enable the measurement of a 3D volumetric reflectivity profile under the raster scanned area. The full volume of interest would be generated by combining measured 3D volumes captured from multiple probe locations/orientations. Since the raster scanning itself maps a 3D volume, fewer points of view would be required to capture the complete 3D volume of interest compared to the point and line scanning methods described above.
Full-field: The OCT probe could use a “full-field OCT” configuration which incorporates imaging optics to simultaneously capture depth reflectivity profiles over a given field-of-view. The full volume of interest would be generated by combining measured 3D volumes captured from multiple probe locations/orientations. Since the full-field implementation itself maps a 3D volume, fewer points of view would be required to capture the complete 3D volume of interest compared to the fixed point and line scanning methods described above.

In some embodiments, the systems herein comprise more than one OCT probe, and/or a means of switching between multiple delivery fibers in a probe so that the OCT is collected from different probe locations without moving the actuator (either in lieu of moving the robot or as an adjunct to it). In that case, which of the probes/fibers was used would also be recorded for each depth capture, so that the known position/orientation of that view can be used to combine each of the captures into a larger 3D volume.

As patients may move slightly relative to the robot arm while scans are taken from different views, a method may be required to ensure that the relative motion of the robot and the patient's teeth are accounted for when multiple volumes are stitched together. This could be accomplished through a number of methods:

The robot arm and OCT probe could be mechanically coupled to the patient's teeth/jaw. This coupling could be rigidly coupled to part of the robot arm that does not move, such that the position of the probe head relative to the rigid coupling can be measured and recorded, allowing the recorded OCT views to be combined based on the probe's location/orientation data alone.

Visual fiducials could be coupled to the patient's teeth in regions to be scanned. These would be used, along with a vision system (camera) in the probe to locate the teeth relative to the probe head. The inferred position of the probe head relative to the fiducials would be used along with the probe's location data locate and orient each of the measured depth profiles in the complete 3D volume capture.

Image registration techniques can be used to combine 3D scans that are partly overlapped by fitting them together in software, without complete a priori knowledge of the point-of-view of the OCT scan. In principle, the 3D image registration can be done without knowing the robot's location/orientation data relative to the teeth, though the robot's location/orientation data could nonetheless also be used as part of the algorithm (to improve speed, reliability, reduce the solution space for the image registration, etc.). The OCT data could also be combined with 3D intraoral scanner data to fill in parts of the 3D volume that are of interest, but invisible to a standard 3D intraoral scanner.

As a whole, the device and methods described above would be capable of imaging the 3D surface of the tooth, including regions that are hidden from direct observation.

In a variation of this concept, confocal laser scanning microscopy (CLSM) would be used in the probe rather than OCT. CLSM, like OCT, is able to measure 3D volumetric images of a sample (albeit with reduced imaging depth and lower depth resolution). CLSM accomplishes this using an optical detection scheme that blocks light not emerging from the microscope's focal plane (which is at a certain depth relative to the microscope's objective/scanning lens).

A number of methods exist to deliver and collect light for CLSM through a fiber or fiber bundle. As such, the distal end of the CLSM system can be incorporated into a probe head similar to that used to deliver fiber-based OCT.

CLSM is inherently a point-scanning method (i.e. it images a single point in 3D space at a time). Depth scanning requires the distal end of the probe to be moved towards or away from the sample (thus moving the focal plane), or otherwise requires an optical setup that can change the distance of the focus from the probe, while maintaining the out-of-focus light rejection of the confocal setup.

Transverse scanning can be done using fixed point, line, or raster scanning, as described above. Once the depth profiles are collected, they can be combined into 3D volumes. Combining such 3D volumes collected from multiple points of view (as described for the OCT case) would allow us to image the entire 3D volume of interest, and to extract the 3D surface information, as required.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. A platform for mapping a three-dimensional (3D) dental anatomy of a subject, the platform comprising:
(a) a non-contact mapping system comprising:
(i) a non-contact sensor capturing a first morphology data of at least a portion of the mouth of the subject; and
(ii) a sensor transmission module transmitting the first morphology data;
(b) a contact mapping system comprising:
(i) a probe comprising:
a probe orientation sensor measuring an orientation of the probe;
a probe contact surface; and
a probe transmission module transmitting the orientation of the probe; and
(ii) a datum comprising:
a datum fastener for mounting to the subject;
a datum orientation sensor measuring an orientation of the datum; and
a probe transmission module transmitting the orientation of the datum; and
(c) a modeling system receiving:
(i) the first morphology data;
(ii) the orientation of the probe; and
(iii) the orientation of the datum;
wherein the modeling system determines a second morphology data based on the orientation of the probe and the orientation of the datum; and
wherein the modeling system determines the 3D dental anatomy based on the first morphology data and the second morphology data.

2. A platform for mapping a three-dimensional (3D) dental anatomy of a subject, the platform comprising:
(a) a non-contact mapping system comprising:
(i) a non-contact sensor capturing a first morphology data of at least a portion of the mouth of the subject; and
(ii) a sensor transmission module transmitting the first morphology data;
(b) a contact mapping system comprising:
(i) a probe comprising:
a probe orientation sensor measuring an orientation of the probe;
a probe contact surface; and
a probe transmission module transmitting the orientation of the probe; and
(c) a modeling system receiving:
(i) the first morphology data; and
(ii) the orientation of the probe;
wherein the modeling system determines the 3D dental anatomy based on the first morphology data and the orientation of the probe.

3. A platform for mapping a three-dimensional (3D) dental anatomy of a subject, the platform comprising:
(a) a non-contact mapping system comprising:
(i) a first non-contact sensor capturing a first portion of a first morphology data of at least a portion of the mouth of the subject;
(ii) a second non-contact sensor capturing a second portion of the first morphology data of at least a portion of the mouth of the subject; and
(iii) a sensor transmission module transmitting the primary first morphology data;
(b) a modeling system receiving:
(i) the first morphology data; and
wherein the modeling system determines the 3D dental anatomy based on the primary first morphology data.

4. The platform of claim 2 or 3, wherein the contact mapping system further comprises a datum comprising a datum fastener for mounting to the subject;

5. The platform of claim 4, wherein the datum further comprises:
(a) a datum orientation sensor measuring an orientation of the datum; and
(b) a probe transmission module transmitting the orientation of the datum.

6. The platform of claim 5, wherein the modeling system further receives the orientation of the datum.

7. The platform of claim 6, wherein the modeling system determines a second morphology data based on the orientation of the probe and the orientation of the datum.

8. The platform of claim 7, wherein the modeling system determines the 3D dental anatomy based on the first morphology data and the second morphology data.

9. The platform of claim 1 or 4, wherein the datum orientation sensor comprises an accelerometer, a tilt sensor, a gyroscope, a GPS sensor, a distance sensor, a RADAR, a magnet, a radio frequency generator, a radio frequency receiver, or any combination thereof.

10. The platform of claim 1 or 4, wherein the datum orientation sensor is located within at most about 2 inches from the center of mass of the datum.

11. The platform of claim 1 or 4, wherein the orientation of the datum comprises a rotation of the datum about one or more axes, a translation of the datum in one or more directions, a rotational velocity of the datum about one or more axes, a translational velocity of the datum in one or more directions, an angular acceleration of the datum about one or more axes, a translational acceleration of the datum in one or more directions, or any combination thereof.

12. The platform of claim 1 or 4, wherein the datum fastener comprises a clip, an adhesive, a clamp, a band, a tie, or any combination thereof

13. The platform of any one of claim 1, 4, or 12, wherein the datum fastener rigidly and removably mounts to the subject.

14. The platform of any one of claim 1, 4, or 10-13, wherein the datum fastener mounts to a tooth of the subject, the jaw of the subject, or both.

15. The platform of any one of claim 1, 4, or 10-13, wherein the datum fastener mounts outside the subject's mouth.

16. The platform of any one of claim 1, 4, or 10-15, wherein the datum further comprises a datum fiducial visible to the non-contact sensor.

17. The platform of claim 16, wherein first morphology data comprises the datum fiducial.

18. The platform of any preceding claim, wherein the non-contact mapping system comprises two or more non-contact sensors.

19. The platform of any preceding claim, wherein the first morphology comprises a visible surface morphology, a subsurface occluded morphology, or both.

20. The platform of claim 19, wherein the subsurface occluded morphology comprises a tooth pulp, a muscle, a nerve, a blood vessel, or any combination thereof.

21. The platform of any preceding claim, wherein the non-contact sensor comprises a 3D scanner, a LIDAR, a RADAR, a laser, a camera, a microscope, an optical coherence tomogram (OCT), a confocal laser scanning microscope (CLSM), or any combination thereof.

22. The platform of claim 21, wherein the OCT comprises a time-domain OCT, a Fourier-domain OCT, a swept-source OCT, or any combination thereof.

23. The platform of claim 21, wherein the camera comprises an endoscopic camera.

24. The platform of claim 23, wherein the endoscopic camera comprises an illumination source.

25. The platform of claim 21, wherein the microscope comprises a confocal laser scanning microscope, a multi-photon microscope, or both.

26. The platform of claim 25, wherein the confocal laser scanning microscope, the multi-photon microscope, or both captures an image of the oral cavity of the subject and a fluorescence within the oral cavity of the subject.

27. The platform of any preceding claim, wherein the non-contact sensor captures the first morphology by point-by-point scanning, line scanning, raster scanning, full-field scanning, or any combination thereof.

28. The platform of any preceding claim, wherein the non-contact mapping system further comprises a switch to initiate the capture of the first morphological data, terminate the capture of the first morphological data, or both.

29. The platform of any preceding claim, wherein the non-contact mapping system further comprises a reference fiducial rigidly and removably mountable to the subject.

30. The platform of claim 29, wherein the non-contact sensor further captures the reference fiducial, and wherein the first morphology data further comprises a location of the reference fiducial, orientation of the reference fiducial, or both.

31. The platform of any preceding claim, wherein the contact mapping system comprises two or more contact sensors.

32. The platform of any preceding claim, wherein the probe orientation sensor comprises an accelerometer, a tilt sensor, a gyroscope, a GPS sensor, a distance sensor, a RADAR, a magnet, a radio frequency generator, a radio frequency receiver, or any combination thereof.

33. The platform of any preceding claim, wherein the probe orientation sensor is located within at most about 2 inches from the center of mass of the probe.

34. The platform of any preceding claim, wherein the orientation of the probe comprises a rotation of the probe about one or more axes, a translation of the probe in one or more directions, a rotational velocity of the probe about one or more axes, a translational velocity of the probe in one or more directions, an angular acceleration of the probe about one or more axes, a translational acceleration of the probe in one or more directions, or any combination thereof.

35. The platform of any one of claims 1, 2, and 4-34, wherein the probe comprises a periodontal endoscope.

36. The platform of any one of claims 1, 2, and 4-35, wherein the probe contact surface comprises a sub-gingival probe contact surface.

37. The platform of any one of claims 1, 2, and 4-36, wherein the probe contact surface is generally acute.

38. The platform of any one of claims 1, 2, and 4-37, wherein the probe contact surface is rounded.

39. The platform of any one of claims 1, 2, and 4-38, wherein at least a portion of the probe contact surface is rigid.

40. The platform of any one of claims 1, 2, and 4-39, wherein at least a portion of the probe contact surface is flexible.

41. The platform of any one of claims 1, 2, and 4-40, wherein at least a portion of the probe contact surface is removable from the probe.

42. The platform of any one of claims 1, 2, and 4-41, wherein the probe further comprises one or more of:
(a) a force sensor measuring a probe force between the probe contact surface and a dental surface of the subject; and
(b) a touch sensor determining if the distal probe contacts the dental surface of the subject.

43. The platform of claim 42, wherein the probe transmission module further transmits the probe force, the contact determination, or both.

44. The platform of claim 42, wherein the modeling system further determines the 3D dental anatomy based on the probe force, the contact determination, or both.

45. The platform of any one of claims 1, 2, and 4-44, wherein a center axis of at least a portion of the probe contact surface is parallel to a center axis of at least a portion of the handle.

46. The platform of any one of claims 1, 2, and 4-45, wherein a center axis of at least a portion of the probe contact surface is askew from a center axis of at least a portion of the handle.

47. The platform of any one of claims 1, 2, and 4-46, wherein the probe further comprises a probe fiducial visible to the non-contact sensor.

48. The platform of claim 47, wherein first morphology data comprises the probe fiducial.

49. The platform of any one of claims 1, 2, and 4-48, wherein the probe further comprises a probe light sensor, and wherein the platform further comprises a pulsed light emitter.

50. The platform of claim 49, wherein a beam of light emitted by the pulsed light emitter translates, rotates, or both, with respect to the probe light sensor.

51. The platform of claim 49 or 50, wherein the probe transmission module further transmits the sensed probe light.

52. The platform of claim 51, wherein the modeling system further determines the 3D dental anatomy based on the sensed probe light.

53. The platform of claim 1 or 4, wherein the datum further comprises a datum light sensor, and wherein the platform further comprises a pulsed light emitter.

54. The platform of claim 53, wherein a beam of light emitted by the pulsed light emitter translates, rotates, or both, with respect to the datum light sensor.

55. The platform of claim 53 or 54, wherein the datum transmission module further transmits the sensed datum light.

56. The platform of claim 55, wherein the modeling system further determines the 3D dental anatomy based on the sensed datum light.

57. The platform of any preceding claim, further comprising an actuator coupled to the non-contact sensor

58. The platform of any one of claims 1, 2, and 4-56, further comprising an actuator coupled to the probe.

59. The platform of claim 57, wherein the non-contact sensor comprises a second coupling that removably connects to the actuator.

60. The platform of claim 57, wherein the non-contact sensor captures a first morphology at a first location, wherein the actuator orients the non-contact sensor to a second location different from the first location, and wherein the non-contact sensor captures a third morphology at the second location.

61. The platform of claim 58, wherein the probe comprises a first coupling that removably connects to the actuator.

62. The platform of any preceding claim, further comprising a encoder measuring a position of the non-contact mapping system

63. The platform of any one of claims 1, 2, and 4-56, further comprising a encoder measuring a position of the probe.

64. The platform of claim 62 or 63, wherein the first morphology is based on a measurement by the passive encoder.

65. The platform of claim 62, wherein the second morphology is based on a measurement by the passive encoder.

66. The platform of claim 62, wherein the encoder is a rotational encoder.

67. The platform of claim 62, wherein the encoder is a translational encoder.

68. The platform of any preceding claim, further comprising a mouth coupling device removably coupling the non-contact mapping to the mouth of the patient.

69. The platform of any preceding claim, wherein the modeling system further determines the 3D dental anatomy based on a historical morphology data of the subject.

70. The platform of any preceding claim, wherein the modeling system normalizes the first morphology data.

71. The platform of claim 70, wherein the first morphology data is normalized by a first machine learning algorithm.

72. The platform of any one of claims 1, 2, 4-56, wherein the modeling system normalizes the second morphology data.

73. The platform of claim 72, wherein the second morphology data is normalized by a first machine learning algorithm

74. The platform of any preceding claim, wherein the modeling system further determines the 3D dental anatomy based on a predetermined anatomy landmark.

75. The platform of claim 74, wherein the modeling system determines the 3D dental anatomy by triangulation, confocal imaging, stereophotogrammetry, or any combination thereof

76. The platform of claim 74, wherein the anatomy landmark comprises a gum margin, a marked tooth, or both.

77. The platform of any preceding claim, wherein the modeling system further determines the 3D dental anatomy by applying a second machine learning algorithm.

78. The platform of any preceding claim, wherein the modeling system further determines an anatomy classification based on the first morphology data and the second morphology data.

79. The platform of claim 78, wherein the anatomy classification comprises a tooth classification, a gum classification, a lip classification, a cheek classification, a tongue classification, or any combination thereof.

80. The platform of claim 78, wherein the modeling system determines the anatomy classification by applying a third machine learning algorithm.

81. The platform of any preceding claim, wherein the modeling system further extrapolates the 3D dental anatomy.

82. The platform of claim 81, wherein the modeling system extrapolates the 3D dental anatomy using a fourth machine learning algorithm.

83. The platform of any preceding claim, wherein the modeling system further interpolates the 3D dental anatomy.

84. The platform of claim 83, wherein the modeling system interpolates the 3D dental anatomy using a fifth machine learning algorithm.

85. The platform of any preceding claim, further comprising a dental effector performing a dental surgery based at least in part on the 3D dental anatomy of the subject.

86. The platform of any preceding claim, wherein the dental anatomy is of a tooth, a jaw, a gum, a lingual tooth surface, a subgingival surface, an interproximal gap, or any combination thereof.

87. The platform of any preceding claim, wherein the first morphology data, the second morphology data, the 3D dental anatomy, or any combination thereof comprises a point cloud, a mesh, a surface model, or any combination thereof.

88. The platform of any one of claims 1 and 3-87, wherein the sensor transmission module, the probe transmission module, the datum transmission module, or any combination thereof comprise a wired transmitter.

89. The platform of any one of claims 1 and 3-87, wherein the sensor transmission module, the probe transmission module, the datum transmission module, or any combination thereof comprise a wireless transmitter.

90. The platform of claim 89, wherein the Bluetooth transmitter, a Wi-Fi transmitter, a cellular transmitter, a wired transmitter, an optical transmitter, or any combination thereof

91. A method for mapping a three-dimensional (3D) dental anatomy of a subject, the method comprising:
(a) capturing, by non-contact sensing, a first morphology data of at least a portion of the mouth of the subject;
(b) transmitting, the first morphology data;
(c) measuring an orientation of a probe while a probe contact surface of the probe contacts the mouth of the subject;
(d) transmitting the orientation of the probe;
(e) measuring an orientation of a datum;
transmitting the orientation of the datum;
(g) determining a second morphology data based on the orientation of the probe and the orientation of the datum; and
(h) determining the 3D dental anatomy based on the first morphology data and the second morphology data.

92. A method for mapping a three-dimensional (3D) dental anatomy of a subject, the method comprising:
(a) capturing, by a non-contact sensor, a first morphology data of at least a portion of the mouth of the subject;
(b) transmitting, by a sensor transmission module, the first morphology data;
(c) measuring, by a probe orientation sensor having a probe contact surface, an orientation of a probe while the probe contact surface contacts the mouth of the subject;
(d) transmitting, by a probe transmission module, the orientation of the probe; and
(e) determining the 3D dental anatomy based on the first morphology data and the orientation of the probe.

93. A method for mapping a three-dimensional (3D) dental anatomy of a subject, the method comprising:
(a) capturing, by a first non-contact sensor, a first portion of a first morphology data of at least a portion of the mouth of the subject;
(b) capturing, by a second non-contact sensor, a second portion of the first morphology data of at least a portion of the mouth of the subject;
(c) transmitting, by a sensor transmission module, the first morphology data;
(d) determining the 3D dental anatomy based on the first morphology data.

94. The method of claim 92, further comprising:
(a) measuring, by a datum orientation sensor, an orientation of the datum; and
(b) transmitting, by a probe transmission module, the orientation of the datum.

95. The method of claim 94, wherein the second morphology data is determined based on the orientation of the probe and the orientation of the datum.

96. The method of claim 95, wherein the 3D dental anatomy is determined based on the first morphology data and the second morphology data.

97. The method of claim 91 or 94, wherein the datum orientation sensor comprises an accelerometer, a tilt sensor, a gyroscope, a GPS sensor, a distance sensor, a RADAR, a magnet, a radio frequency generator, a radio frequency receiver, or any combination thereof.

98. The method of claim 91 or 94, wherein the datum orientation sensor is located within at most about 2 inches from the center of mass of the datum.

99. The method of claim 91 or 94, wherein the orientation of the datum comprises a rotation of the datum about one or more axes, a translation of the datum in one or more directions, a rotational velocity of the datum about one or more axes, a translational velocity of the datum in one or more directions, an angular acceleration of the datum about one or more axes, a translational acceleration of the datum in one or more directions, or any combination thereof.

100. The method of claim 91 or 94, further comprising mounting the datum to the subject with a datum fastener.

101. The method of claim 100, wherein mounting the datum to the subject comprises rigidly and removably mounting the datum to the subject.

102. The method of claim 100, wherein mounting the datum to the subject comprises mounting the datum to a tooth of the subject, the jaw of the subject, or both.

103. The method of claim 100, wherein mounting the datum to the subject comprises mounting the datum outside the subject's mouth.

104. The method of any preceding claim, further comprising measuring, by an encoder, a position of the non-contact mapping system.

105. The method of claim 104, wherein the first morphology is based on a measurement by the passive encoder.

106. The method of any one of claim 91, 92, or 94, further comprising measuring, by an encoder, a position of the probe.

107. The method of claim 106, wherein the second morphology is based on a measurement by the passive encoder

108. The method of claim 104, wherein the encoder is a rotational encoder.

109. The method of claim 104, wherein the encoder is a translational encoder.

110. The method of any one of claim 91, 94, or 100-103, further comprising capturing, by the non-contact sensor, a datum fiducial on the datum.

111. The method of claim 104, wherein first morphology data comprises the datum fiducial.

112. The method of any preceding claim, wherein the non-contact sensor comprises a 3D scanner, a LIDAR, a RADAR, a laser, a camera, a microscope, an optical coherence tomogram (OCT), a confocal laser scanning microscope (CLSM), or any combination thereof

113. The method of claim 112, wherein the OCT comprises a time-domain OCT, a Fourier-domain OCT, a swept-source OCT, or any combination thereof.

114. The method of claim 112, wherein the camera comprises an endoscopic camera.

115. The method of claim 114, wherein the endoscopic camera comprises an illumination source.

116. The method of claim 112, wherein the microscope comprises a confocal laser scanning microscope, a multi-photon microscope, or both.

117. The method of claim 112, wherein the confocal laser scanning microscope, the multi-photon microscope, or both, capture an image of a fluorescence within the oral cavity of the subject.

118. The method of claim 117, further comprising applying the fluorescence to the oral cavity of the subject.

119. The method of any preceding claim, wherein the first morphology comprises a visible surface morphology, a subsurface occluded morphology, or both.

120. The method of claim 119, wherein the subsurface occluded morphology comprises a tooth pulp, a muscle, a nerve, a blood vessel, or any combination thereof.

121. The method of any preceding claim, wherein capturing the first morphology comprises point-by-point scanning, line scanning, raster scanning, full-field scanning, or any combination thereof.

122. The method of any preceding claim, wherein capturing the first morphology comprises activating a switch to initiate the capture of the first morphological data, terminate the capture of the first morphological data, or both.

123. The method of any preceding claim, further comprising mounting a reference fiducial to the subject.

124. The method of claim 123, wherein the first morphology data further comprises a location of the reference fiducial, orientation of the reference fiducial, or both.

125. The method of any preceding claim, wherein the probe orientation sensor comprises an accelerometer, a tilt sensor, a gyroscope, a GPS sensor, a distance sensor, a RADAR, a magnet, a radio frequency generator, a radio frequency receiver, or any combination thereof.

126. The method of any preceding claim, wherein the probe orientation sensor is located within at most about 2 inches from the center of mass of the probe.

127. The method of any preceding claim, wherein the orientation of the probe comprises a rotation of the probe about one or more axes, a translation of the probe in one or more directions, a rotational velocity of the probe about one or more axes, a translational velocity of the probe in one or more directions, an angular acceleration of the probe about one or more axes, a translational acceleration of the probe in one or more directions, or any combination thereof.

128. The method of any preceding claim, wherein the probe comprises a periodontal endoscope.

129. The method of any preceding claim, wherein the probe contact surface comprises a sub-gingival probe contact surface.

130. The method of any preceding claim, wherein the probe contact surface is generally acute.

131. The method of any preceding claim, wherein the probe contact surface is rounded.

132. The method of any preceding claim, wherein at least a portion of the probe contact surface is rigid.

133. The method of any preceding claim, wherein at least a portion of the probe contact surface is flexible.

134. The method of any preceding claim, further comprising one or more of:
(a) measuring, by a force sensor, a probe force between the probe contact surface and a dental surface of the subject; and
(b) determining, by a touch sensor, if the distal probe contacts the dental surface of the subject.

135. The method of claim 134, further comprising transmitting, by the transmission module, the probe force, the contact determination, or both.

136. The method of claim 134, wherein the 3D dental anatomy is further based on the probe force, the contact determination, or both.

137. The method of any preceding claim, wherein a center axis of at least a portion of the probe contact surface is parallel to a center axis of at least a portion of the handle.

138. The method of any preceding claim, wherein a center axis of at least a portion of the probe contact surface is askew from a center axis of at least a portion of the handle.

139. The method of any preceding claim, wherein the probe further comprises a probe fiducial visible to the non-contact sensor.

140. The method of claim 139, wherein first morphology data comprises the probe fiducial.

141. The method of any preceding claim, wherein the probe further comprises a probe light sensor, and wherein the method further comprises emitting a light beam by a pulsed light emitter.

142. The method of claim 141, further comprising translating the light beam, rotating the light beam, or both, with respect to the probe light sensor.

143. The method of claim 141 or 142, further comprising transmitting, by the probe transmission module, the sensed probe light.

144. The method of claim 143, wherein 3D dental anatomy is further based on the sensed probe light.

145. The method of claim 91 or 94, wherein the datum further comprises a datum light sensor, and wherein the method further comprises emitting a light beam by a pulsed light emitter.

146. The method of claim 145, further comprising translating the light beam, rotating the light beam, or both, with respect to the datum light sensor.

147. The method of claim 145 or 146, further comprising transmitting, by the datum transmission module, the sensed datum light.

148. The method of claim 147, wherein the 3D dental anatomy is further based on the sensed datum light.

149. The method of any preceding claim, further comprising orienting, by an actuator, the non-contact mapping system.

150. The method of claim 149, further comprising capturing the first morphology at a first location, orienting, by the actuator, the non-contact sensor to a second location different from the first location, and capturing, by the non-contact sensor, a third morphology at the second location.

151. The method of claim, 91 or 92, further comprising orienting, by an actuator, the probe.

152. The method of any preceding claim, further comprising coupling the non-contact mapping sensor to the mouth of the subject.

153. The method of any preceding claim, wherein the 3D dental anatomy is further based on a historical morphology data of the subject.

154. The method of any preceding claim, further comprising normalizing the first morphology data.

155. The method of claim 154, wherein the first morphology data is normalized by a first machine learning algorithm.

156. The method of claim 91 or 92, further comprising normalizing the second morphology data.

157. The method of claim 156, wherein the second morphology data is normalized by a first machine learning algorithm.

158. The method of any preceding claim, wherein the 3D dental anatomy is further based on a predetermined anatomy landmark.

159. The method of claim 158, wherein the 3D dental anatomy is determined by triangulation, confocal imaging, stereophotogrammetry, or any combination thereof

160. The method of claim 158, wherein the anatomy landmark comprises a gum margin, a marked tooth, or both.

161. The method of any preceding claim, wherein the 3D dental anatomy is determined by applying a second machine learning algorithm.

162. The method of any preceding claim, further comprising determining an anatomy classification based on the first morphology data and the second morphology data.

163. The method of claim 162, wherein the anatomy classification comprises a tooth classification, a gum classification, a lip classification, a cheek classification, a tongue classification, or any combination thereof.

164. The method of any preceding claim, further comprising extrapolating the 3D dental anatomy.

165. The method of claim 164, wherein extrapolating the 3D dental anatomy is performed by a fifth machine learning algorithm.

166. The method of any preceding claim, wherein the modeling system further interpolates the 3D dental anatomy.

167. The method of claim 166, wherein the modeling system interpolates the 3D dental anatomy using a fourth machine learning algorithm.

168. The method of any preceding claim, further comprising performing a dental surgery based at least in part on the 3D dental anatomy of the subject.

169. The method of any preceding claim, wherein the dental anatomy is of a tooth, a jaw, a gum, a lingual tooth surface, a subgingival surface, an interproximal gap, or any combination thereof.

170. The method of any preceding claim, wherein the first morphology data, the second morphology data, the 3D dental anatomy, or any combination thereof comprises a point cloud, a mesh, a surface model, or any combination thereof

171. The method of any preceding claim, wherein the sensor transmission module, the probe transmission module, the datum transmission module, or any combination thereof comprise a wired transmitter.

172. The method of any preceding claim, wherein the sensor transmission module, the probe transmission module, the datum transmission module, or any combination thereof comprise a wireless transmitter.

173. The method of claim 172, wherein the Bluetooth transmitter, a Wi-Fi transmitter, a cellular transmitter, a wired transmitter, an optical transmitter, or any combination thereof
